SEQUENCE LISTING (111 sequences)

SEQ ID NO: 1 - DNA, 23 bp, Artificial Sequence
  Synthetic polynucleotide
tacgtgaagcaccccgccgacat

SEQ ID NO: 2 - DNA, 21 bp, Artificial Sequence
  Synthetic polynucleotide
ttcttgtaatcggggatgtcg

SEQ ID NO: 3 - DNA, 20 bp, Artificial Sequence
  Synthetic polynucleotide
cccgccgacatccccgatta

SEQ ID NO: 4 - DNA, 57 bp, Artificial Sequence
  Synthetic polynucleotide
tacgtgaagcaccccgccgacatccccgattttcttgtaatcggggatgtcggcggg

SEQ ID NO: 5 - DNA, 59 bp, Artificial Sequence
  Synthetic polynucleotide
tacgtgaagcaccccgccgacatccccgattacttcttgtaatcggggatgtcggcggg

SEQ ID NO: 6 - DNA, 60 bp, Artificial Sequence
  Synthetic polynucleotide
tacgtgaagcaccccgccgacatccccgattacgttcttgtaatcggggatgtcggcggg

SEQ ID NO: 7 - DNA, 61 bp, Artificial Sequence
  Synthetic polynucleotide
tacgtgaagcaccccgccgacatccccgattactgttcttgtaatcggggatgtcggcggg

SEQ ID NO: 8 - DNA, 62 bp, Artificial Sequence
  Synthetic polynucleotide
tacgtgaagcaccccgccgacatccccgattactgattcttgtaatcggggatgtcggcggg

SEQ ID NO: 9 - DNA, 63 bp, Artificial Sequence
  Synthetic polynucleotide
tacgtgaagcaccccgccgacatccccgattactgacttcttgtaatcggggatgtcggcggg

SEQ ID NO: 10 - DNA, 65 bp, Artificial Sequence
  Synthetic polynucleotide
tacgtgaagcaccccgccgacatccccgattactgactgttcttgtaatcggggatgtcggcggg

SEQ ID NO: 11 - DNA, 67 bp, Artificial Sequence
  Synthetic polynucleotide
tacgtgaagcaccccgccgacatccccgattactgactgacttcttgtaatcggggatgtcggcggg

SEQ ID NO: 12 - DNA, 68 bp, Artificial Sequence
  Synthetic polynucleotide
tacgtgaagcaccccgccgacatccccgattactgactgacgttcttgtaatcggggatgtcggcggg

SEQ ID NO: 13 - DNA, 73 bp, Artificial Sequence
  Synthetic polynucleotide
tacgtgaagcaccccgccgacatccccgattactgactgactgactgttcttgtaatcggggatgtcggcggg

SEQ ID NO: 14 - skipped

SEQ ID NO: 15 - DNA, 57 bp, Artificial Sequence
  Synthetic polynucleotide
aatcggggatgtcggcggggtgcttcacgtacccgccgacatccccgattacaagaa

SEQ ID NO: 16 - DNA, 59 bp, Artificial Sequence
  Synthetic polynucleotide
aatcggggatgtcggcggggtgcttcacgtaaccccgccgacatccccgattacaagaa

SEQ ID NO: 17 - DNA, 60 bp, Artificial Sequence
  Synthetic polynucleotide
aatcggggatgtcggcggggtgcttcacgtaacgcccgccgacatccccgattacaagaa

SEQ ID NO: 18 - DNA, 61 bp, Artificial Sequence
  Synthetic polynucleotide
aatcggggatgtcggcggggtgcttcacgtaactgcccgccgacatccccgattacaagaa

SEQ ID NO: 19 - DNA, 62 bp, Artificial Sequence
  Synthetic polynucleotide
aatcggggatgtcggcggggtgcttcacgtaactgacccgccgacatccccgattacaagaa

SEQ ID NO: 20 - DNA, 63 bp, Artificial Sequence
  Synthetic polynucleotide
aatcggggatgtcggcggggtgcttcacgtaactgaccccgccgacatccccgattacaagaa

SEQ ID NO: 21 - DNA, 65 bp, Artificial Sequence
  Synthetic polynucleotide
aatcggggatgtcggcggggtgcttcacgtaactgactgcccgccgacatccccgattacaagaa

SEQ ID NO: 22 - DNA, 67 bp, Artificial Sequence
  Synthetic polynucleotide
aatcggggatgtcggcggggtgcttcacgtaactgactgaccccgccgacatccccgattacaagaa

SEQ ID NO: 23 - DNA, 68 bp, Artificial Sequence
  Synthetic polynucleotide
aatcggggatgtcggcggggtgcttcacgtaactgactgacgcccgccgacatccccgattacaagaa

SEQ ID NO: 24 - DNA, 73 bp, Artificial Sequence
  Synthetic polynucleotide
aatcggggatgtcggcggggtgcttcacgtaactgactgactgactgcccgccgacatccccgattacaagaa

SEQ ID NO: 25 - skipped

SEQ ID NO: 26 - DNA, 57 bp, Artificial Sequence
  Synthetic polynucleotide
tacgtgaagcaccccgccgacatccccgattcccgccgacatccccgattacaagaa

SEQ ID NO: 27 - DNA, 59 bp, Artificial Sequence
  Synthetic polynucleotide
tacgtgaagcaccccgccgacatccccgattaccccgccgacatccccgattacaagaa

SEQ ID NO: 28 - DNA, 60 bp, Artificial Sequence
  Synthetic polynucleotide
tacgtgaagcaccccgccgacatccccgattacgcccgccgacatccccgattacaagaa

SEQ ID NO: 29 - DNA, 61 bp, Artificial Sequence
  Synthetic polynucleotide
tacgtgaagcaccccgccgacatccccgattactgcccgccgacatccccgattacaagaa

SEQ ID NO: 30 - DNA, 62 bp, Artificial Sequence
  Synthetic polynucleotide
tacgtgaagcaccccgccgacatccccgattactgacccgccgacatccccgattacaagaa

SEQ ID NO: 31 - DNA, 63 bp, Artificial Sequence
  Synthetic polynucleotide
tacgtgaagcaccccgccgacatccccgattactgaccccgccgacatccccgattacaagaa

SEQ ID NO: 32 - DNA, 65 bp, Artificial Sequence
  Synthetic polynucleotide
tacgtgaagcaccccgccgacatccccgattactgactgcccgccgacatccccgattacaagaa

SEQ ID NO: 33 - DNA, 67 bp, Artificial Sequence
  Synthetic polynucleotide
tacgtgaagcaccccgccgacatccccgattactgactgaccccgccgacatccccgattacaagaa

SEQ ID NO: 34 - DNA, 68 bp, Artificial Sequence
  Synthetic polynucleotide
tacgtgaagcaccccgccgacatccccgattactgactgacgcccgccgacatccccgattacaagaa

SEQ ID NO: 35 - DNA, 73 bp, Artificial Sequence
  Synthetic polynucleotide
tacgtgaagcaccccgccgacatccccgattactgactgactgactgcccgccgacatccccgattacaagaa

SEQ ID NO: 36 - skipped

SEQ ID NO: 37 - DNA, 59 bp, Artificial Sequence
  Synthetic polynucleotide
ttcttgtaatcggggatgtcggcggggtaatcggggatgtcggcggggtgcttcacgta

SEQ ID NO: 38 - DNA, 61 bp, Artificial Sequence
  Synthetic polynucleotide
ttcttgtaatcggggatgtcggcggggtacaatcggggatgtcggcggggtgcttcacgta

SEQ ID NO: 39 - DNA, 62 bp, Artificial Sequence
  Synthetic polynucleotide
ttcttgtaatcggggatgtcggcggggtacgaatcggggatgtcggcggggtgcttcacgta

SEQ ID NO: 40 - DNA, 63 bp, Artificial Sequence
  Synthetic polynucleotide
ttcttgtaatcggggatgtcggcggggtactgaatcggggatgtcggcggggtgcttcacgta

SEQ ID NO: 41 - DNA, 64 bp, Artificial Sequence
  Synthetic polynucleotide
ttcttgtaatcggggatgtcggcggggtactgaaatcggggatgtcggcggggtgcttcacgta

SEQ ID NO: 42 - DNA, 65 bp, Artificial Sequence
  Synthetic polynucleotide
ttcttgtaatcggggatgtcggcggggtactgacaatcggggatgtcggcggggtgcttcacgta

SEQ ID NO: 43 - DNA, 67 bp, Artificial Sequence
  Synthetic polynucleotide
ttcttgtaatcggggatgtcggcggggtactgactgaatcggggatgtcggcggggtgcttcacgta

SEQ ID NO: 44 - DNA, 69 bp, Artificial Sequence
  Synthetic polynucleotide
ttcttgtaatcggggatgtcggcggggtactgactgacaatcggggatgtcggcggggtgcttcacgta

SEQ ID NO: 45 - DNA, 70 bp, Artificial Sequence
  Synthetic polynucleotide
ttcttgtaatcggggatgtcggcggggtactgactgacgaatcggggatgtcggcggggtgcttcacgta

SEQ ID NO: 46 - DNA, 75 bp, Artificial Sequence
  Synthetic polynucleotide
ttcttgtaatcggggatgtcggcggggtactgactgactgactgaatcggggatgtcggcggggtgcttcacgta

SEQ ID NO: 47 - skipped

SEQ ID NO: 48 - DNA, 59 bp, Artificial Sequence
  Synthetic polynucleotide
accccgccgacatccccgattacaagaatacgtgaagcaccccgccgacatccccgatt

SEQ ID NO: 49 - DNA, 61 bp, Artificial Sequence
  Synthetic polynucleotide
accccgccgacatccccgattacaagaaactacgtgaagcaccccgccgacatccccgatt

SEQ ID NO: 50 - DNA, 62 bp, Artificial Sequence
  Synthetic polynucleotide
accccgccgacatccccgattacaagaaacgtacgtgaagcaccccgccgacatccccgatt

SEQ ID NO: 51 - DNA, 63 bp, Artificial Sequence
  Synthetic polynucleotide
accccgccgacatccccgattacaagaaactgtacgtgaagcaccccgccgacatccccgatt

SEQ ID NO: 52 - DNA, 64 bp, Artificial Sequence
  Synthetic polynucleotide
accccgccgacatccccgattacaagaaactgatacgtgaagcaccccgccgacatccccgatt

SEQ ID NO: 53 - DNA, 65 bp, Artificial Sequence
  Synthetic polynucleotide
accccgccgacatccccgattacaagaaactgactacgtgaagcaccccgccgacatccccgatt

SEQ ID NO: 54 - DNA, 67 bp, Artificial Sequence
  Synthetic polynucleotide
accccgccgacatccccgattacaagaaactgactgtacgtgaagcaccccgccgacatccccgatt

SEQ ID NO: 55 - DNA, 69 bp, Artificial Sequence
  Synthetic polynucleotide
accccgccgacatccccgattacaagaaactgactgactacgtgaagcaccccgccgacatccccgatt

SEQ ID NO: 56 - DNA, 70 bp, Artificial Sequence
  Synthetic polynucleotide
accccgccgacatccccgattacaagaaactgactgacgtacgtgaagcaccccgccgacatccccgatt

SEQ ID NO: 57 - DNA, 75 bp, Artificial Sequence
  Synthetic polynucleotide
accccgccgacatccccgattacaagaaactgactgactgactgtacgtgaagcaccccgccgacatccccgatt

SEQ ID NO: 58 - skipped

SEQ ID NO: 59 - DNA, 59 bp, Artificial Sequence
  Synthetic polynucleotide
ttcttgtaatcggggatgtcggcggggttacgtgaagcaccccgccgacatccccgatt

SEQ ID NO: 60 - DNA, 61 bp, Artificial Sequence
  Synthetic polynucleotide
ttcttgtaatcggggatgtcggcggggtactacgtgaagcaccccgccgacatccccgatt

SEQ ID NO: 61 - DNA, 62 bp, Artificial Sequence
  Synthetic polynucleotide
ttcttgtaatcggggatgtcggcggggtacgtacgtgaagcaccccgccgacatccccgatt

SEQ ID NO: 62 - DNA, 63 bp, Artificial Sequence
  Synthetic polynucleotide
ttcttgtaatcggggatgtcggcggggtactgtacgtgaagcaccccgccgacatccccgatt

SEQ ID NO: 63 - DNA, 64 bp, Artificial Sequence
  Synthetic polynucleotide
ttcttgtaatcggggatgtcggcggggtactgatacgtgaagcaccccgccgacatccccgatt

SEQ ID NO: 64 - DNA, 65 bp, Artificial Sequence
  Synthetic polynucleotide
ttcttgtaatcggggatgtcggcggggtactgactacgtgaagcaccccgccgacatccccgatt

SEQ ID NO: 65 - DNA, 67 bp, Artificial Sequence
  Synthetic polynucleotide
ttcttgtaatcggggatgtcggcggggtactgactgtacgtgaagcaccccgccgacatccccgatt

SEQ ID NO: 66 - DNA, 69 bp, Artificial Sequence
  Synthetic polynucleotide
ttcttgtaatcggggatgtcggcggggtactgactgactacgtgaagcaccccgccgacatccccgatt

SEQ ID NO: 67 - DNA, 70 bp, Artificial Sequence
  Synthetic polynucleotide
ttcttgtaatcggggatgtcggcggggtactgactgacgtacgtgaagcaccccgccgacatccccgatt

SEQ ID NO: 68 - DNA, 75 bp, Artificial Sequence
  Synthetic polynucleotide
ttcttgtaatcggggatgtcggcggggtactgactgactgactgtacgtgaagcaccccgccgacatccccgatt

SEQ ID NO: 69 - skipped

SEQ ID NO: 70 - DNA, 54 bp, Artificial Sequence
  Synthetic polynucleotide
ttcttgtaatcggggatgtcggcggggtttcttgtaatcggggatgtcggcggg

SEQ ID NO: 71 - DNA, 56 bp, Artificial Sequence
  Synthetic polynucleotide
ttcttgtaatcggggatgtcggcggggtacttcttgtaatcggggatgtcggcggg

SEQ ID NO: 72 - DNA, 57 bp, Artificial Sequence
  Synthetic polynucleotide
ttcttgtaatcggggatgtcggcggggtacgttcttgtaatcggggatgtcggcggg

SEQ ID NO: 73 - DNA, 58 bp, Artificial Sequence
  Synthetic polynucleotide
ttcttgtaatcggggatgtcggcggggtactgttcttgtaatcggggatgtcggcggg

SEQ ID NO: 74 - DNA, 59 bp, Artificial Sequence
  Synthetic polynucleotide
ttcttgtaatcggggatgtcggcggggtactgattcttgtaatcggggatgtcggcggg

SEQ ID NO: 75 - DNA, 60 bp, Artificial Sequence
  Synthetic polynucleotide
ttcttgtaatcggggatgtcggcggggtactgacttcttgtaatcggggatgtcggcggg

SEQ ID NO: 76 - DNA, 62 bp, Artificial Sequence
  Synthetic polynucleotide
ttcttgtaatcggggatgtcggcggggtactgactgttcttgtaatcggggatgtcggcggg

SEQ ID NO: 77 - DNA, 64 bp, Artificial Sequence
  Synthetic polynucleotide
ttcttgtaatcggggatgtcggcggggtactgactgacttcttgtaatcggggatgtcggcggg

SEQ ID NO: 78 - DNA, 65 bp, Artificial Sequence
  Synthetic polynucleotide
ttcttgtaatcggggatgtcggcggggtactgactgacgttcttgtaatcggggatgtcggcggg

SEQ ID NO: 79 - DNA, 70 bp, Artificial Sequence
  Synthetic polynucleotide
ttcttgtaatcggggatgtcggcggggtactgactgactgactgttcttgtaatcggggatgtcggcggg

SEQ ID NO: 80 - skipped

SEQ ID NO: 81 - DNA, 54 bp, Artificial Sequence
  Synthetic polynucleotide
accccgccgacatccccgattacaagaacccgccgacatccccgattacaagaa

SEQ ID NO: 82 - DNA, 56 bp, Artificial Sequence
  Synthetic polynucleotide
accccgccgacatccccgattacaagaaaccccgccgacatccccgattacaagaa

SEQ ID NO: 83 - DNA, 57 bp, Artificial Sequence
  Synthetic polynucleotide
accccgccgacatccccgattacaagaaacgcccgccgacatccccgattacaagaa

SEQ ID NO: 84 - DNA, 58 bp, Artificial Sequence
  Synthetic polynucleotide
accccgccgacatccccgattacaagaaactgcccgccgacatccccgattacaagaa

SEQ ID NO: 85 - DNA, 59 bp, Artificial Sequence
  Synthetic polynucleotide
accccgccgacatccccgattacaagaaactgacccgccgacatccccgattacaagaa

SEQ ID NO: 86 - DNA, 60 bp, Artificial Sequence
  Synthetic polynucleotide
accccgccgacatccccgattacaagaaactgaccccgccgacatccccgattacaagaa

SEQ ID NO: 87 - DNA, 62 bp, Artificial Sequence
  Synthetic polynucleotide
accccgccgacatccccgattacaagaaactgactgcccgccgacatccccgattacaagaa

SEQ ID NO: 88 - DNA, 64 bp, Artificial Sequence
  Synthetic polynucleotide
accccgccgacatccccgattacaagaaactgactgaccccgccgacatccccgattacaagaa

SEQ ID NO: 89 - DNA, 65 bp, Artificial Sequence
  Synthetic polynucleotide
accccgccgacatccccgattacaagaaactgactgacgcccgccgacatccccgattacaagaa

SEQ ID NO: 90 - DNA, 70 bp, Artificial Sequence
  Synthetic polynucleotide
accccgccgacatccccgattacaagaaactgactgactgactgcccgccgacatccccgattacaagaa

SEQ ID NO: 91 - skipped

SEQ ID NO: 92 - DNA, 54 bp, Artificial Sequence
  Synthetic polynucleotide
ttcttgtaatcggggatgtcggcggggtcccgccgacatccccgattacaagaa

SEQ ID NO: 93 - DNA, 56 bp, Artificial Sequence
  Synthetic polynucleotide
ttcttgtaatcggggatgtcggcggggtaccccgccgacatccccgattacaagaa

SEQ ID NO: 94 - DNA, 57 bp, Artificial Sequence
  Synthetic polynucleotide
ttcttgtaatcggggatgtcggcggggtacgcccgccgacatccccgattacaagaa

SEQ ID NO: 95 - DNA, 58 bp, Artificial Sequence
  Synthetic polynucleotide
ttcttgtaatcggggatgtcggcggggtactgcccgccgacatccccgattacaagaa

SEQ ID NO: 96 - DNA, 59 bp, Artificial Sequence
  Synthetic polynucleotide
ttcttgtaatcggggatgtcggcggggtactgacccgccgacatccccgattacaagaa

SEQ ID NO: 97 - DNA, 60 bp, Artificial Sequence
  Synthetic polynucleotide
ttcttgtaatcggggatgtcggcggggtactgaccccgccgacatccccgattacaagaa

SEQ ID NO: 98 - DNA, 62 bp, Artificial Sequence
  Synthetic polynucleotide
ttcttgtaatcggggatgtcggcggggtactgactgcccgccgacatccccgattacaagaa

SEQ ID NO: 99 - DNA, 64 bp, Artificial Sequence
  Synthetic polynucleotide
ttcttgtaatcggggatgtcggcggggtactgactgaccccgccgacatccccgattacaagaa

SEQ ID NO: 100 - DNA, 65 bp, Artificial Sequence
  Synthetic polynucleotide
ttcttgtaatcggggatgtcggcggggtactgactgacgcccgccgacatccccgattacaagaa

SEQ ID NO: 101 - DNA, 70 bp, Artificial Sequence
  Synthetic polynucleotide
ttcttgtaatcggggatgtcggcggggtactgactgactgactgcccgccgacatccccgattacaagaa

SEQ ID NO: 102 - protein, 972 aa, Artificial Sequence
  Synthetic polypeptide
MYPYDVPDYAGPKKKRKVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVALRGSGGGSGGGSGGGSGGGSGGGSGGGSVLLNVLSKCAGSKKFRPAPAAAFARECRGFYFELQELKEDDYYGITLSDDSDHQFLLANQVVVHNCTMTEKGSGGRADALDDFDLDMLGSDALDDFDLDMLGSDALDDFDLDMLGSDALDDFDLDMLINC

SEQ ID NO: 103 - protein, 1016 aa, Artificial Sequence
  Synthetic polypeptide
MDYKDDDDKPKKKRKVSRPGERPFQCRICMRNFSDKTKLRVHTRTHTGEKPFQCRICMRNFSVRHNLTRHLRTHTGEKPFQCRICMRNFSQSTSLQRHLKTHLRGFGGVLEKGCFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGCHVRKATYQTYAPIGGGSGGGSGGGSGGGSGGGSGGGSTQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVA

SEQ ID NO: 104 - protein, 870 aa, Artificial Sequence
  Synthetic polypeptide
MYPYDVPDYAGPKKKRKVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVALRGSGGGSGGGSGGGSGGGSGGGSGGGSVLLNVLSKCAGSKKFRPAPAAAFARECRGFYFELQELKEDDYYGITLSDDSDHQFLLANQVVVHNCTMTEKGSGGRADALDDFDLDMLGSDALDDFDLDMLGSDALDDFDLDMLGSDALDDFDLDMLINC

SEQ ID NO: 105 - protein, 1118 aa, Artificial Sequence
  Synthetic polypeptide
MDYKDDDDKPKKKRKVSRPGERPFQCRICMRNFSDKTKLRVHTRTHTGEKPFQCRICMRNFSVRHNLTRHLRTHTGEKPFQCRICMRNFSQSTSLQRHLKTHLRGFGGVLEKGCFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGCHVRKATYQTYAPIGGGSGGGSGGGSGGGSGGGSGGGSTQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVA

SEQ ID NO: 106 - protein, 1257 aa, Artificial Sequence
  Synthetic polypeptide
MYPYDVPDYAGSLAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSMRGSGGGSGGGSGGGSGGGSGGGSGGGSVLLNVLSKCAGSKKFRPAPAAAFARECRGFYFELQELKEDDYYGITLSDDSDHQFLLANQVVVHNCTMTEKGSGGRADALDDFDLDMLGSDALDDFDLDMLGSDALDDFDLDMLGSDALDDFDLDMLINC

SEQ ID NO: 107 - protein, 1414 aa, Artificial Sequence
  Synthetic polypeptide
MDYKDDDDKPKKKRKVSRPGERPFQCRICMRNFSDKTKLRVHTRTHTGEKPFQCRICMRNFSVRHNLTRHLRTHTGEKPFQCRICMRNFSQSTSLQRHLKTHLRGFGGVLEKGCFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGCHVRKATYQTYAPIGGGSGGGSGGGSGGGSGGGSGGGSTLMAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKV

SEQ ID NO: 108 - protein, 1453 aa, Artificial Sequence
  Synthetic polypeptide
MDYKDDDDKPKKKRKVSRPGERPFQCRICMRNFSDKTKLRVHTRTHTGEKPFQCRICMRNFSVRHNLTRHLRTHTGEKPFQCRICMRNFSQSTSLQRHLKTHLRGFGGVLEKGCFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGCHVRKATYQTYAPIGGGSGGGSGGGSGGGSGGGSGGGSTLMSDLVLGLAIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVAAILPLSITFDDSLANKVLVYATAAQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINDKGKEVPCNPFLKYKEEHGYIRKYSKKGNGPEIKSLKYYDSKLGNHIDITPKDSNNKVVLQSVSPWRADVYFNKTTGKYEILGLKYADLQFDKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDFSRADPKKKRKV

SEQ ID NO: 109 - protein, 1263 aa, Artificial Sequence
  Synthetic polypeptide
MYPYDVPDYAGSLAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKVMRGSGGGSGGGSGGGSGGGSGGGSGGGSVLLNVLSKCAGSKKFRPAPAAAFARECRGFYFELQELKEDDYYGITLSDDSDHQFLLANQVVVHNCTMTEKGSGGRADALDDFDLDMLGSDALDDFDLDMLGSDALDDFDLDMLGSDALDDFDLDMLINC

SEQ ID NO: 110 - protein, 1408 aa, Artificial Sequence
  Synthetic polypeptide
MDYKDDDDKPKKKRKVSRPGERPFQCRICMRNFSDKTKLRVHTRTHTGEKPFQCRICMRNFSVRHNLTRHLRTHTGEKPFQCRICMRNFSQSTSLQRHLKTHLRGFGGVLEKGCFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGCHVRKATYQTYAPIGGGSGGGSGGGSGGGSGGGSGGGSTLLAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGS

SEQ ID NO: 111 - DNA, 352 bp, Artificial Sequence
  Synthetic polynucleotide
tgaagaagctgcaggaggtgctggaggggaagtggtccggatcttgaagaagctgcaggaggtgctggaggggaagtggtccggatcttgaagaagctgcaggaggtgctggaggggaagtggtccggatcttgaagaagctgcaggaggtgctggaggggaagtggtccggatcttgaagaagctgcaggaggtgctggaggggaagtggtccggatcttgaagaagctgcaggaggtgctggaggggaagtggtccggatcttgaagaagctgcaggaggtgctggaggggaagtggtccggatcttgaagaagctgcaggaggtgctggaggggaagtggtccggatct

(19) United States
(12) Patent Application Publication
(10) Pub. No.: US 2021/0189485 A1
(43) Pub. Date: Jun. 24, 2021

(54) SEQUENCE DETECTION SYSTEMS

(71) Applicant: The Jackson Laboratory, Bar Harbor, ME (US)

(72) Inventors: Albert Cheng, Bar Harbor, ME (US); Aziz Taghbalout, Bristol, CT (US); Nathaniel Lee Jillette, Bar Harbor, ME (US)

(73) Assignee: The Jackson Laboratory, Bar Harbor, ME (US)

(21) Appl. No.: 16/761,298

(22) PCT Filed: Nov. 6, 2018
(86) PCT No.: PCT/US2018/059334

§ 371 Date: May 4, 2020

Related U.S. Application Data
(60) Provisional application No. 62/581,903, filed on Nov. 6, 2017.

(51) Int. Cl. C12Q 1/6876; C12N 9/22; C12N 15/11; C12Q 1/6869

(52) U.S. Cl. CPC C12Q 1/6876; C12N 9/22; C12N 2800/80; C12Q 1/6869; C12N 2310/20; C12N 15/11

(57) ABSTRACT

The present disclosure, in some embodiments, provides sequence detection systems (sequence detectors) for the detection of specific nucleotides sequences present in the genome of live cells (e.g., single live cells) to achieve, for example, in vivo and in situ imaging, cell selection, and/or cell ablation.

### RELATED APPLICATIONS

This application claims the benefit under 35 U.S.C. § 119(e) of U.S. provisional application No. 62/581,903, filed Nov. 6, 2017, which is incorporated by reference herein in its entirety.

### FEDERALLY-SPONSORED RESEARCH

This invention was made with government support under grant number P30CA034196 awarded by National Institutes of Health. The government has certain rights in the invention.

### BACKGROUND

The ability to identify populations of live cells with changes to chromosome sequences introduced by programmed DNA editing, mutations in the DNA sequences, or aberrant chromosomal rearrangements is tremendously advantageous to research investigations. Tools for selecting these populations of live cells include zinc finger (ZF) DNA sensors that are able to detect (sense) a specific DNA sequence and effectively report upon its detection by producing a detectable (e.g., fluorescent) signal (see, e.g., Slomovic S. & Collins J. Nature Methods 2015; 12(11): 1085-1092). These ZF DNA sensors rely on the cumbersome assembly of ZF pairs specific to each targeted sequence, and the specificity and affinity of the artificial ZFs requires screening and validation using in vitro and in vivo approaches.

### SUMMARY

The present disclosure provides, in some aspects, sequence detection systems (sequence detectors) that may enable early diagnostic and preventative medicine as well as a way to track genomic evolution in vivo. The technology provided herein is developed to detect, in some embodiments, cancer-specific sequences present in the genome of live cells (e.g., single live cells) to achieve, for example, in vivo and in situ imaging, cell selection, and/or cell ablation. By coupling these sequence detectors to a response circuitry, a particular cellular program can be triggered upon sequence detection to achieve therapeutic functions. For example, malignant cells can be specifically induced to self-destruct upon acquiring a particular genetic aberration. The basis of sequence detection enables, inter alia, personalized precision medicine tailored to each defined genetic sequence.

The sequence detectors provided herein use programmable DNA-binding pair modules (e.g., catalytically inactive orthogonal Cas9 nucleases) to enable detection of specific non-repeat sequences that ZF DNA sensors failed to detect. Further, the sequence detectors of the present disclosure, relative to ZF DNA sensors, are more specific, more effective, and versatile.

Some aspects of the present disclosure provide sequence detector systems comprising (a) a first guide RNA (gRNA) and a first catalytically-inactive RNA-guided nuclease linked to an N-terminal fragment of an intein, wherein the N-terminal fragment is linked to a first polypeptide, and the first gRNA is engineered to bind to a first target sequence, and (b) a second gRNA and a second catalytically-inactive RNA-guided nuclease linked to an C-terminal fragment of an intein, wherein the C-terminal fragment is linked to a second polypeptide, and the second gRNA is engineered to bind to a second target sequence adjacent to the first target sequence, wherein the first and second catalytically-inactive RNA-guided nucleases are orthogonal to each other. In some embodiments, the N-terminal fragment and the C-terminal fragment of the intein catalyze joining of the first polypeptide to the second polypeptide.

Other aspects of the present disclosure provide a pair of engineered polynucleotides, wherein the first polynucleotide of the pair encodes in the 5′ to 3′ direction a first polypeptide, an N-terminal fragment of an intein, a first catalytically-inactive RNA-guided nuclease, and optionally a first guide RNA (gRNA) engineered to bind to a first target sequence, and the second polynucleotide of the pair encodes in the 5′ to 3′ direction a second catalytically-inactive RNA-guided nuclease, a C-terminal fragment of the intein, and a second polypeptide, and optionally a second gRNA engineered to bind to a second target sequence adjacent to the first target sequence.

In some embodiments, the first and second catalytically-inactive RNA-guided nucleases are selected from catalytically-inactive Cas9 nucleases and catalytically-inactive Cpf1 nucleases. For example, the first and second catalytically-inactive RNA-guided nucleases may be selected from catalytically-inactive Streptococcus thermophiles, Staphylococcus aureus, and Neisseria meningitidis Cas9 nucleases. In some embodiments, the first catalytically-inactive Cas9 nuclease is a catalytically-inactive Streptococcus thermophiles Cas9 nuclease and the second catalytically-inactive Cas9 nuclease is a catalytically-inactive Neisseria meningitidis Cas9 nuclease.

Further aspects of the present disclosure provide sequence detector systems comprising (a) a first TAL effector DNA-binding domain (TALE) linked to an N-terminal fragment of an intein, wherein the N-terminal fragment is linked to a first polypeptide, and the first TALE is engineered to bind to a first target sequence, and (b) a second TALE linked to an C-terminal fragment of an intein, wherein the C-terminal fragment is linked to a second polypeptide, and the second TALE is engineered to bind to a second target sequence adjacent to the first target sequence.

Additional aspects of the present disclosure provide a pair of engineered polynucleotides, wherein the first polynucleotide of the pair encodes in the 5′ to 3′ direction a first polypeptide, an N-terminal fragment of an intein, and a first TAL effector DNA-binding domain (TALE) engineered to bind to a first target sequence, and the second polynucleotide of the pair encodes in the 5′ to 3′ direction a second TALE engineered to bind to a second target sequence adjacent to the first target sequence, a C-terminal fragment of the intein, and a second polypeptide.

In some embodiments, a first and/or second polynucleotide is present on an expression vector, optionally a DNA plasmid.

In some embodiments, the intein is an engineered split intein or a naturally-occurring split intein. For example, the intein may be selected from Saccharomyces cerevisiae VMA (See VMA) split inteins, Synechocystis sp. DnaB (Ssp DnaB) split inteins, Synechocystis sp. GyrB (Ssp GyrB) split inteins, Synechocystis sp. DnaE (Ssp DnaE) split inteins, and Nostoc punctiforme DnaE (Npu DnaE) split inteins.

In some embodiments, (a) the first polypeptide is a first reporter molecule and the second polypeptide is a second reporter molecule; or (b) the first polypeptide is an N-terminal fragment of a reporter molecule and the second polypeptide is a C-terminal fragment of the reporter molecule. In some embodiments, the first and/or second reporter molecule of (a) and/or the reporter molecule of (b) is selected from TagCFP, mTagCFP2, Azurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOκ, mKO2, mOrange, mOrange2, mRaspberry, mCherry, mStrawberry, mScarlet, mTangerine, tdTomato, TagRFP, TagRFP-T, mApple, mRuby, mRuby2, mPlum, HcRed-Tandem, mKate2, mNeptune, NirFP, TagRFP657, IFP1.4 and iRFP.

In some embodiments, the first and second reporter molecules of (a) are different from each other.

In some embodiments, the first polypeptide is an N-terminal fragment of a toxic molecule and the second polypeptide is a C-terminal fragment of the toxic molecule. In some embodiments, the toxic molecule is selected from toxins, pro-apoptotic proteins, and prodrug metabolic enzymes

In some embodiments, the first polypeptide is a first molecule of a synthetic transcription factor and the second polypeptide is a second molecule of the synthetic transcription factor; or the first polypeptide is an N-terminal fragment of a synthetic transcription factor and the second polypeptide is a C-terminal fragment of the synthetic transcription factor. In some embodiments, the synthetic transcription factor binds to and activates transcription of a nucleic acid encoding a reporter molecule or a toxic molecule. In some embodiments, the nucleic acid encoding a reporter molecule or a toxic molecule comprises a minimal promoter and a binding site to which the synthetic transcription factor binds.

In some embodiments, the N terminus of the first catalytically-inactive RNA-guided nuclease is linked to the C terminus of the N-terminal fragment of the intein, the N terminus of the N-terminal fragment of the intein is linked to the C terminus of the first polypeptide, the C terminus of the second catalytically-inactive RNA-guided nuclease is linked to the N terminus of the C-terminal fragment of the intein, and the C terminus of the C-terminal fragment of the intein is linked to the N terminus of the second polypeptide.

Also provided herein are cells comprising (a) a sequence detector system or a pair of engineered polynucleotides and (b) a genome comprising the first and second target sequences. In some embodiments, the first target sequence and the second target sequence are separated from each by fewer than 25 nucleotides. In some embodiments, the cell is a live cancer cell, optionally in vitro, in situ, or in vivo. In some embodiments, the first and second target sequences are cancer-specific target sequences.

Further provided herein are selective detection methods comprising delivering to a population of cells a pair of engineered polynucleotides of the present disclosure, and assaying for expression or activity of the reporter molecule.

Further provided herein are cell ablation methods comprising delivering to a population of cells the pair of engineered polynucleotides of the present disclosure, and assaying for cell death.

In some embodiments, the population of cells comprises cancer cells, and wherein the first and second target sequences are specific to the cancer cells.

### BRIEF DESCRIPTION OF THE DRAWINGS

FIGS. 1A-1C depict strategies for sequence detectors. FIG. 1A shows that two DNA binding proteins fused to different fluorescent proteins can be programmed to bind to 5′ and 3′ junctional sequences of defined genomic rearrangement events. WT cells have two disparate foci while cells with gene fusion have overlapping fluorescent foci. FIG. 1B shows two DNA binding proteins can tether halves of a split fluorescent protein that can be reconstituted based on intein-mediated protein splicing, eliciting signals in cells with the fused gene. FIG. 1C shows sensor-based reconstitution of a toxin can trigger cell death specifically in cells with fused genes.

FIGS. 2A-2B show an overview of CRISPR/Cas9-based sequence detectors (CRISPR.sense). FIG. 2A is an illustration of a ST1-Nm dCas9-based sequence detectors. The indicated dCas9 orthologues and their gRNA serve as DNA-binding pair modules mediating DNA sequence recognition of the associated sequence detectors. The target sequences for CRISPR.sense systems were designed as a single copy (1×) within a replicative plasmid. The configuration of the PAM sequences and gaps separating the dCas9 binding sequences are shown. Intein-based trans-splicing transducer system and GFP-based reporter plasmid are the same for the dCas9-based sequence detector and ZF-based DNA sensor. FIG. 2B is a schematic representation of alternative CRISPR.sense tested using the indicated combinations of dCas9 orthologues and their respective sgRNAs. The configuration of the intein-based transducer linked to the indicated dCas9 is the same within all the four CRISPR-based sequence detectors.

FIGS. 3A-3D show fluorescent activated cell sorting (FACS) analyses of cells transfected with the ZF DNA sensor components or with CRISPR.sense components using indicated dCas9-based sequence detectors and corresponding target substrates comprising the shown PAM configuration and gap size. There were eight (8) binding sites within the replicative target plasmid for the ZF DNA sensor, and there was one (1) binding site for the dCas9-based sequence detector. FIG. 3A shows dCas9-based sequence detector-1 (Nm-VmaCt-VP64/ZF9-VmaNt-ST1), FIG. 3B shows Cas9-based sequence detector-2 (Sa-VmaCt-VP64/ZF9-VmaNt-Nm), FIG. 3C shows dCas9-based sequence detector-3 (Sa-VmaCt-VP64/ZF9-VmaNt-ST1), and FIG. 3D shows dCas9-based sequence detector-4 (Nm-VmaCt-VP64/ZF9-VmaNt-Sa).

FIGS. 4A-4B describe the TALE-based sequence detector (TALE.Sense). FIG. 4A is a schematic representation of sequence detectors based on TALE DNA-binding modules (left). Bipartite sequence targets and gaps in base pair (bp) separating each binding site are shown. The target sequences are present in 8 copies (8×) on a replicative plasmid. Intein-based transducer includes a N-terminal split of SceVma intein fused to the carboxyl end ZF9, and a SceVma intein C-terminal split fused to the amino terminal end of a transcription activator VP64. Reconstitution of intein, mediated by binding of DNA-binding module pair to target sites, leads to the trans-splicing of a response module ZF9-VP64. The reporter includes a plasmid containing coding sequence for GFP placed down-stream of a minimal promoter and six ZF9 binding sites as indicated. Binding of the reported module mediated by ZF9 and ZF9-operator leads to expression of GFP that can be recorded by using a flow cytometer as illustrated in the column plot shown at the top. FIG. 4B shows FACS analysis of cells transfected with ZF-based DNA sensor, or TALE-based sequence detector using target sequences with the indicated gap size. TALE DNA-binding modules were engineered to bind the left side (TALE 1L) or right side (TALE 1R) of the bipartite target sequences.

FIGS. 5A-5E show structural requirements for TALE-based sequence detector. FIG. 5A shows a schematic representation of intein-mediated trans-splicing of the response module leading to activation of GFP expression. FIG. 5B and FIG. 5D depict the structure of TALE DNA-binding pair modules of the TALE-based sequence detectors and target sequences used to transfect cells analyzed in the plots shown in FIG. 5C and FIG. 5E respectively. The gap size is indicated according to a ZF DNA sensor.

FIGS. 6A-6B show the detection of non-repeat sequences. Comparison of a ZF DNA sensor and TALE-based sequence detector-1 in their efficiency to report on a non-repeat target sequence of a non-replicative plasmid. Because the gap size requirement for a ZF DNA sensor and a TALE-based sequence detector are different, template with no gap (optimal for ZF-based DNA sensor) or 8 bp gap (optimal for TALE-based sequence detectors) were tested. Drawings in FIG. 6A depict the TALE-based sequence detector and targets used to transfect cells analyzed by FACS in FIG. 6B. The gap size is indicated according to the ZF DNA sensor system.

### DETAILED DESCRIPTION

The present disclosure provides sequence detector systems that detect and report on the presence of specific nucleotide sequences of interest (target sequences) and are based on programmable DNA binding events. These sequence detector systems (sequence detectors) include a pair of modules, and each module includes (a) a programmable DNA-binding domain (e.g., dCas9/gRNA) that “detects” a target sequence linked to (b) a polypeptide (e.g., reporter molecule or toxic molecule) that “reports” on that detection.

The sequences detectors described herein may be used to detect target sequences in vitro, in situ, and/or in vivo. In some embodiments, target sequence is a sequence associated with or indicative of a particular disease (e.g., cancer).

### RNA-Guided Nucleases

In some embodiments, the present disclosure provides a sequence detector comprising: (a) a first guide RNA (gRNA) and a first catalytically-inactive RNA-guided nuclease linked to an N-terminal fragment of an intein, wherein the N-terminal fragment is linked to a first polypeptide, and the first gRNA is engineered to bind to a first target sequence, and (b) a second gRNA and a second catalytically inactive RNA-guided nuclease linked to a C-terminal fragment of an intein, wherein the C-terminal fragment is linked to a second polypeptide, and the second gRNA is engineered to bind to a second target sequence adjacent to the first target sequence, and wherein the first and second catalytically-inactive RNA-guided nucleases are orthogonal to each other.

A guide RNA (gRNA) is a short, synthetic RNA with a scaffold sequence and a spacer sequence. The scaffold sequence binds a RNA-guided nuclease (e.g., Cas or Cpf1), and the spacer sequence binds to a target sequence. See Jinek et al., Science, 337, 816-821 (2012) and Deltcheva et al., Nature, 471, 602-607 (2011). Thus, a gRNA directs the binding of a RNA-guided nuclease to a target sequence. Guide RNAs can be engineered to bind a target sequence (e.g., in a nucleotide sequence in a genome). In some embodiments, gRNAs are recombinantly produced by expressing gRNA sequences in test tubes by in vitro transcription or in cells from a different organism (e.g., bacteria such as Escherichia coli and/or yeast such as Saccharomyces cerevisiae).

In some embodiments, the spacer sequence of a gRNA has a length of 15 to 30 nucleotides. In some embodiments, the spacer sequence has a length of 15, 16, 17, 18, 19, 29, 21, 22, 23, 24, 25, 26, 27, 28, 29, or 30 nucleotide base pairs. In some embodiments, a spacer sequence has a length of 20 nucleotides.

In some embodiments, the total length of a gRNA is 40 to 80 nucleotides. In some embodiments, the total length of a gRNA is at least at least 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 105 nucleotides, 110 nucleotides, 115 nucleotides, or 120 nucleotides long.

Multiple gRNAs can be utilized to guide the binding of RNA-guided nucleases to more than one target sequence. In some embodiments, a first gRNA is engineered to bind to a first target sequence and a second gRNA is engineered to bind to a second target sequence. These target sequences, in some embodiments, are adjacent to each other. For example, a first target sequence and a second target sequence may be located within 1 to 100 nucleotides (nucleotide base pairs) from each other. That is, 1 to 100 nucleotides may be located between the first target sequence and the second target sequences. In some embodiments, 1 to 5, 1 to 10, 1 to 20, 1 to 30, 1 to 40, 1 to 50, 5 to 10, 5 to 20, 5to 30, 5 to 40, 5 to 50, 10 to 20, 10 to 30, 10 to 40, or 10 to 50 nucleotides are located between the first and second target sequences. In some embodiments, 1, 2, 3, 4, 5, 6, 7, 8, 9, 10, 11, 12, 13, 14, 15, 16, 17, 18, 19, or 20 nucleotides are located between the first and second target sequences.

In some embodiments, a gRNA is expressed and produced in a cell that comprises a target sequence (e.g., a sequence indicative of cancer) in its genome. For example, a nucleic acid encoding a gRNA sequence may be cloned into an expression vector (e.g., comprising a promoter and other genetic elements required for transcription), which is then delivered to a cell. A vector is a DNA molecule used to artificially transmit genetic material (e.g., gRNA) into a cell, where it can be replicated or expressed. Non-limiting examples of vectors include plasmids, cosmids, phages and viral vectors.

RNA-guided nucleases are guided to a target sequence by a gRNA. Non-limiting examples of RNA-guided nucleases include Clustered Regularly Interspaced Palindromic Repeats-Associated (CRISPR/Cas) nucleases (e.g., Cas9 nucleases), RNA-guided FokI-nucleases (RFNs), and Cpf1 nucleases.

CRISPR/Cas nucleases exist in a variety of bacterial species, where they recognize and cut specific sequences in the DNA. The CRISPR/Cas nucleases are grouped into two classes. Class 1 systems use a complex of multiple CRISPR/Cas proteins to bind and degrade nucleic acids, whereas Class 2 systems use a large, single protein for the same purpose. A CRISPR/Cas nuclease used herein may be selected from Cas9, Cas10, Cas3, Cas4, C2c1, C2C3, Cas13a, Cas13b, Cas13c, and Cas14 (e.g., Harrington L B et al. Science 2018 (DOI: 10.1126/science.aav4294)). CRISPR/Cas nucleases from different bacterial species have different properties (e.g., specificity, activity, binding affinity). In some embodiments, orthogonal RNA-guided nuclease species are used. Orthogonal species are distinct species (e.g., two or more bacterial species). For example, a first catalytically-inactive Cas9 (dCas9) nuclease used herein may be a Streptococcus thermophilus dCas9 and a second catalytically-inactive Cas9 nuclease used herein may be a Neisseria meningitidis dCas9.

Non-limiting examples of bacterial and archaeal CRISPR/Cas nucleases for use in sequence detector systems of the present disclosure include Streptococcus thermophilus Cas9, Streptococcus thermopilus Cas10, Streptococcus thermophilus Cas3, Staphylococcus aureus Cas9, Staphylococcus aureus Cas10, Staphylococcus aureus Cas3, Neisseria meningitidis Cas9, Neisseria meningitidis Cas10, Neisseria meningitidis Cas3, Streptococcus pyogenes Cas9, Streptooccus pyogenes Cas10, and Streptococcus pyogenes Cas3.

In some embodiments, a RNA-guided nuclease is a RNA-guided FokI nuclease (RFN). FokI nucleases are bacterial endonucleases with an N-terminal DNA-binding domain and a C-terminal endonuclease domain. The DNA-binding domain binds to a 5′-GGATG-3′ target sequence, after which the endonuclease domain cleaves in a non-sequence specific manner. RNA-guided FokI-nuclease (RFN) is a fusion protein derived from catalytically-inactive Streptococcus pyogenes Cas9 protein fused to the FokI nuclease domain. A fusion protein is a protein that includes at least two domains that are encoded by separate genes that have been joined so that they are transcribed and translated as a single unit, producing a single polypeptide. In some embodiments, a catalytically-inactive RNA-guided nuclease is a RNA-guided Fok1 nuclease (RFN), which has greater DNA-binding specificity due to the Cas9 protein than FokI nuclease.

In some embodiments, a RNA-guided nuclease is CRISPR-associated endonuclease in Prevotella and Francisella 1 (Cpf1). Cpf1 is a bacterial endonuclease similar to Cas9 nuclease in terms of activity. However, Cpf1 only requires a short (˜42-nucleotide) gRNA, while Cas9 requires a longer (˜100 nucleotide) gRNA. Additionally, Cpf1 cuts the DNA 5′ to the target sequence and leaves staggered, single-stranded overhangs, whereas Cas9 cuts the DNA 3′ to the target sequence and leaves blunted ends. Cpf1 proteins from Acidaminococcus and Lachnospiraceae bacteria efficiently cut DNA in human cells in vitro. In some embodiments, the RNA-guided nuclease is Acidaminococcus Cpf1 or Lachnospiraceae Cpf1, which require shorter gRNAs than Cas nuclease proteins.

In some embodiments, a RNA-guided nuclease is a catalytically-inactive RNA-guided nuclease. Catalytically-inactive RNA-guided nucleases are RNA-guided nucleases in which the nuclease binds a gRNA and its target sequence, but does not cut the nucleic acid (the catalytic domain is inactive). A RNA-guided nuclease can be catalytically inactivated by deletion of a portion of the polypeptide sequence or by mutation of one or more amino acid residues that are critical for catalytic activity. Catalytically-inactive RNA-guided nucleases can be utilized to bind specific target sequences in a genome without cutting the sequence.

In some embodiments, a catalytically inactive RNA-guided nuclease is an endonuclease dead Cas (dCas) protein. In some embodiments, a dCas protein is dCas9. Cas9 nuclease contains two endonuclease domains (e.g., RuvC and HNH domains). The point mutations D10A and H840A result in deactivation of Cas9 activity. In some embodiments, a catalytically inactive RNA-guided nuclease is an endonuclease dead Fok1 (dFok1) protein. The point mutation D450A results in deactivation of Fok1 activity. In some embodiments, a catalytically-inactive RNA guided nuclease is an endonuclease dead Cpf1 (dCpf1) protein. In some embodiments, a dCpf1 protein is Acidoaminococcus Cpf1 (AsdCpf1). The point mutation D908A results in deactivation of Cpf1 activity.

In some embodiments, the first and second catalytically-inactive RNA guided-nucleases are selected from cataytically-inactive Cas9 nucleases and catalytically inactive Cpf1 nucleases. In some embodiments, the first and second catalytically-inactive RNA-guided nucleases are selected from catalytically inactive Streptococcus thermophilus, Staphylococcus aureus, and Neisseria meningitidis Cas9 nucleases. In some embodiments, the first catalytically-inactive Cas9 nuclease is a catlytically-inactive Streptococcus thermophilus Cas9 nuclease and the second catalytically-inactive Cas9 nuclease is a catalytically-inactive Nesisseria meningitidis Cas9 nuclease.

In some embodiments, a catalytically-inactive RNA-guided nuclease is linked to a molecule to guide the molecule to a specific target sequence. If two catalytically-inactive RNA-guided nucleases are linked to fragments of the same molecule and the target sequences of the two catalytically-inactive RNA-guided nucleases are adjacent, then the binding of the catalytically-inactive RNA-guided nucleases will promote the fusion of the two molecule fragments.

### Transcription Activator Like-Effectors

In some embodiments, a sequence detector system comprises: a first transcription activator like-effector DNA-binding domain (TALE) linked to an N-terminal fragment of an intein, wherein the N-terminal fragment is linked to a first polypeptide, and the first TALE is engineered to bind to a first target sequence, and a second TALE linked to a C-terminal fragment of an intein, wherein the C-terminal fragment is linked to a second polypeptide, and the second TALE is engineered to bind to a second target sequence adjacent to the first target sequence.

Transcription activator-like effectors (TALEs) found in bacteria are modular DNA binding domains that include central repeat domains made up of repetitive sequences of residues (Boch J. et al. Annual Review of Phytopathology 2010; 48: 419-36; Boch J Biotechnology 2011; 29(2): 135-136). The central repeat domains, in some embodiments, contain between 1.5 and 33.5 repeat regions, and each repeat region may be made of 34 amino acids; amino acids 12 and 13 of the repeat region, in some embodiments, determines the nucleotide specificity of the TALE and are known as the repeat variable diresidue (RVD) (Moscou M J et al. Science 2009; 326 (5959): 1501; Juillerat A et al. Scientific Reports 2015; 5: 8150). Unlike ZF DNA sensors, TALE-based sequence detectors can recognize single nucleotides. In some embodiments, combining multiple repeat regions produces sequence-specific synthetic TALEs (Cermak T et al. Nucleic Acids Research 2011; 39 (12): e82).

In some embodiments, a first TALE is engineered to bind to a first target sequence and a second TALE is engineered to bind to a second target sequence. These target sequences, in some embodiments, are adjacent to each other. For example, a first target sequence and a second target sequence may be located within 1 to 100 nucleotides (nucleotide base pairs) from each other. That is, 1 to 100 nucleotides may be located between the first target sequence and the second target sequences. In some embodiments, 1 to 5, 1 to 10, 1 to 20, 1 to 30, 1 to 40, 1 to 50, 5 to 10, 5 to 20, 5to 30, 5 to 40, 5 to 50, 10 to 20, 10 to 30, 10 to 40, or 10 to 50 nucleotides are located between the first and second target sequences. In some embodiments, 1, 2, 3, 4, 5, 6, 7, 8, 9, 10, 11, 12, 13, 14, 15, 16, 17, 18, 19, or 20 nucleotides are located between the first and second target sequences.

### Inteins

An intein (intervening protein) is a polypeptide sequence embedded in a precursor protein that carries out a unique auto-processing event known as protein splicing, in which it excises itself out form the larger precursor polypeptide through the cleavage of two peptide bonds and, in the process, ligates the flanking extein (external protein) sequences through the formation of a new peptide bond. Intein-mediated protein splicing is spontaneous because it requires no external factor or energy source, but relies on the folding of the intein domain. The precursor protein contains three segments—an N-extein (N-terminal portion of the precursor protein), followed by the intein, followed by a C-extein (C-terminal portion of the precursor protein). Following intein splicing, the N-extein is linked to the C-extein.

In some embodiments, the intein is an engineered split intein or a naturally-occurring split intein. Split inteins are separate polypeptides that mediate protein splicing after the intein fragments and their polypeptide cargo associate (see, e.g., Paulus, H Annu Rev Biochem 69:447-496 (2000); and Saleh L, Perler F B Chem Rec 6:183-193 (2006)). Split inteins catalyze a series of chemical rearrangements that require the intein to be properly folded and assembled. The first step in splicing involves an N—S acyl shift in which the N-extein polypeptide is transferred to the side chain of the first residue of the intein. This is then followed by a trans-(thio)esterification reaction in which this acyl unit is transferred to the first residue of the C-extein (which is serine, threonine, or cysteine) to form a branched intermediate. This branched intermediate is then cleaved from the intein by a transamidation reaction involving the C-terminal asparagine residue of the itein. Finally, a S—N acyl transfer occurs to create a normal peptide bond between the two remaining exteins (Lockless, S W, Muir T W, PNAS 106(27): 10999-11004 (2009)).

To date, there are at least 70 different intein alleles, distinguished not only by the type of host gene in which the inteins are embedded, but also the integration point within that host gene (Perler, F B Nucleic Acids Res. 30: 383-384 (2002); Piertrokovski, S Trends Genet. 17: 465-472 (2001)). A small fraction (less than 5%) of the identified intein genes encode split inteins. Unlike contiguous inteins, split inteins are transcribed and translated as two separate polypeptides, the N-intein and C-intein, each linked to one extein. Upon translation, the intein fragments spontaneously and non-covalently assembly (cooperatively fold) into the canonical intein structure to carry out the protein splicing in trans. The first two split inteins to be characterized, from the cyanobacteria Syncheocystis species PCC6803 (Ssp) and Nostoc punctiforme PCC73102 (Npu), are orthologs naturally found inserted in the alpha-subunit of DNA Polymerase III (DnaE). Npu is especially notable due to its remarkably fast rate of protein trans-splicing (t_{1/2}=50 s at 30° C.). This half-life is significantly shorter than that of Ssp (t_{1/2}=80 min at 30° C.) (Shah, N H et al. J. Am. Chem. Soc. 135: 5839 (2013)).

Herein, split inteins are used, in some embodiments, to catalyze the joining of two fragments (e.g., an N-terminal fragment and a C-terminal fragment) of a detectable proteins, such as a fluorescent protein, to produce a functional, full-length protein. A split intein may be a natural split intein or an engineered split intein. Natural split inteins naturally occur in a variety of different organisms. The largest known family of split inteins is found with the DnaE genes of at least 20 cyanobacterial species (Caspi J., et al. Mol. Microbiol. 50: 1569-1577 (2003)). Thus, in some embodiments of the present disclosure, a natural split intein is selected from DnaE inteins. Non-limiting examples of DnaE inteins include Synechocstis sp. DnaE (Ssp DnaE) inteins and Nostoc punctiforme (NpuDnaE) inteins. In some embodiments the present disclosure, a natural split intein is selected from vacuolar ATPase subunit (VMA) inteins. Non-limiting examples of VMA include Saccharomyces cerevisiae VMA inteins.

In some embodiments, a split intein is an engineered split intein. Engineered split inteins are artificially produced and may be produced from contiguous inteins (where a contiguous intein is artificially split) or may be modified natural split inteins that, for example, promote efficient protein purification, ligation, modification, and cyclization (e.g., Npu_{GEP} and Cfa_{GEP}, as described by Stevens, A J PNAS 114(32): 8538-8543 (2017)). Methods for engineering split inteins are described, for example, by Aranko, A S et al. Protein Eng Des Sel. 27(8) 263-271 (2014), incorporated herein by reference. In some embodiments, the engineered split intein is engineered from DnaB inteins (Wu, H, et al. Biochim Biophys Acta 1387 (1-2): 422-432 (1998)). For example, the engineered split intein may be a Ssp DnaB S1 intein. In some embodiments, the engineered split intein is engineered from GyrB inteins. For example, the engineered split intein may be a SspGyrB S11 intein.

In some embodiments, the intein is selected from Saccharomyces cerevisiae VMA (See VMA) split inteins, Synechocystis sp. DnaB (Ssp DnaB) split inteins, Synechocystis sp. GyrB (Ssp GyrB) split inteins, Synechocystis sp. DnaE (Ssp DnaE) split inteins, and Nostoc punctiforme DnaE (Npu DnaE) split inteins.

Catalytically-inactive RNA-guided nucleases can be utilized to promote the joining of split intein fragments. In some embodiments, the N-terminus of the first catalytically inactive RNA-guided nuclease is linked to the C-terminus of the N-terminal fragment of an intein, and wherein the N-terminus of the N-terminal fragment of the molecule is linked to the C-terminus of a first polypeptide, and wherein the C-terminus of the second catalytically-inactive RNA-guided nuclease is linked to the N-terminus of the C-terminal fragment of the intein, and wherein the C-terminus of the C-terminal fragment of the intein is linked to the N-terminus of the second polypeptide.

In some embodiments, the N-terminus of the first TALE is linked to the C-terminus of the N-terminal fragment of the intein, and the N-terminus of the N-terminal fragment of the intein is linked to the C-terminus of the first polypeptide, and the C-terminus of the second TALE is linked to the C-terminal fragment of the intein, and the C-terminus of the C-terminal fragment of the intein is linked to the N-terminus of the second polypeptide.

### Polypeptides

A polypeptide is a polymer of (two or more) amino acid residues. Polypeptides of the present disclosure generally form molecules that function to provide a detectable signal indicative of binding of a sequence detector to a specific target sequence. Non-limiting examples of these molecules include reporter molecules, a toxic molecules, synthetic transcription factors. The polypeptides may be fragments of a full-length peptide or protein (each fragment linked to a split intein fragment, for example), or a polypeptide itself may be a full-length peptide or protein. For example, a first polypeptide may be the N-terminal fragment of Protein X (e.g., N-terminal GFP) and the second polypeptide may be the C-terminal fragment of Protein X (e.g., C-terminal GFP) such that when the first and second polypeptides are joined (e.g., fused) a functional Protein X (e.g., GFP) is produced. As another example, a first polypeptide may be a functional full-length Protein X (e.g., full-length GFP) and the second polypeptide may be functional full-length Protein Y (e.g., full-length RFP).

Linkage of protein fragments to intein fragments facilitates protein splicing, in some embodiments, to produce full-length functional protein (e.g., fluorescent protein).

Reporter Molecules

A reporter molecule is a molecule that produces a signal (e.g., a visible or otherwise detectable signal) when the molecule is expressed or activated. A reporter molecule may be a protein or a nucleic acid. In some embodiments, the first polypeptide is a first reporter molecule and the second polypeptide is a second reporter molecule. In some embodiments, the first polypeptide is one fragment (e.g., N-terminal fragment) of a reporter molecule and the second polypeptide is another fragment (e.g., C-terminal fragment) of a reporter molecule. In some embodiments, the first and second polypeptide, when joined (e.g., through intein-mediated protein splicing), form a synthetic transcription factor that activates transcription of a nucleic acid encoding reporter molecule (e.g., encoded on a separate plasmid).

In some embodiments, a reporter molecule is a fluorescent protein that fluoresces at an appropriate wavelength of light when expressed either in vitro or in vivo. Non-limiting examples of fluorescent proteins include GFP, EGFP, Emerald, Superfolder GFP, Azami Green, mWasabi, TagGFP, TurboGFP, AcGFP, ZsGreen, T-Sapphire, EBFP, EBFP2, Azurite, mTagBFP, ECFP, mECFP, Cerulean, mTurquoise, CyPet, AmCyan1, Midori-Ishi Cyan, TagCFP, mTFP1 (Teal), EYFP, Topaz, Venus, mCitrine, YPet, TagYFP, PhiYFP, ZsYellow1, mBanana, Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, mRuby, mApple, mStrawberry, AsRed2, mRFP1, JRed, mCherry, HcRed1, mRaspberry, dKeima-Tandem, HcRed-Tandem, mPlum, AQ143, mKalam1, Sirius, SCFP3C, Czami Green, mUKG, Clover, mNeonGreen, SYFP2, mKOκ, mKO2, mScarlet, mRuby, mRuby2, mKate2, mNeptune, NirFP, TagRFP657, IFP1.4, and iRFP.

In some embodiments, the first reporter molecule is a first fluorescent protein and the second reporter molecule is a second fluorescent protein, wherein the first fluorescent protein is different from the second fluorescent protein.

In some embodiments, a first polypeptide and a second polypeptide encode fragments of a single reporter molecule. In some embodiments, the first polypeptide is an N-terminal fragment of a reporter molecule and the second polypeptide is a C-terminal fragment of the reporter molecule.

Toxic Molecules

A toxic molecule is a molecule that induces cell death (cell ablation) when the molecule is expressed or activated. Cell ablation refers to selectively destroying cells in which the reporter toxic molecule is expressed. In some embodiments, the first polypeptide is a first toxic molecule and the second polypeptide is a second toxic molecule. In some embodiments, the first polypeptide is one fragment (e.g., N-terminal fragment) of a toxic molecule and the second polypeptide is another fragment (e.g., C-terminal fragment) of a toxic molecule. In some embodiments, the first and second polypeptide, when joined (e.g., through intein-mediated protein splicing), form a synthetic transcription factor that activates transcription of a nucleic acid encoding toxic molecule (e.g., encoded on a separate plasmid). Non-limiting examples of toxic molecules include toxins, pro-apoptotic proteins and prodrug metabolic enzymes. In some embodiments, the toxic molecules include the NTR-CB 1954 pair, wherein the toxicity of CB 1954 (5-(aziridin-1-yl)-2,4-dinitrobenzamide) is dependent upon its reduction by a bacterial nitroreductase (NTR), which transforms it into an agent of DNA inter-strand cross-linking and apoptosis (PMID: 8375021). In some embodiments, the toxic molecule is herpes simplex virus thymidine kinase (HSV-TK), which converts ganciclovir (GCV) into a toxic product and allows selective elimination of TK+ cells (Blankenstein et al. Human Gene Therapy 2008; 6(12)).

Non-limiting examples of toxins include Corynebacterium diptheriae diptheria toxin, Escherichia colizEF toxin, viral protein M2(H37A), lipopolysaccharide (LPS), lipooligosaccharide (LOS), Clostiridum botulinum toxin, Clostridium tetani toxin, Bordatella pertussis toxin, Staphylococcus aureus Exoliatin B toxin, Bacillus anthracis toxin, Pseudomonas aeruoginosa exotoxin, and Shigella dysenteriae toxin.

Synthetic Transcription Factors

A synthetic transcription factor is a protein with a DNA binding domain and a transcription activator domain that increases the transcriptional activity of a gene or a set of genes. The DNA binding domain binds to a sequence near the promoter of a gene, and the activator domain binds to and recruits other proteins and transcription factors active in gene transcription. The gene transcribed may produce a reporter molecule or a toxic molecule. In some embodiments, the first polypeptide is one fragment (e.g., N-terminal fragment) of a synthetic transcription factor and the second polypeptide is another fragment (e.g., C-terminal fragment) of a synthetic transcription factor. In some embodiments, the first and second polypeptide, when joined (e.g., through intein-mediated protein splicing), form a synthetic transcription factor that activates transcription of a nucleic acid (e.g., a reporter gene) encoding a reporter molecule or a toxic molecule (e.g., encoded on a separate plasmid). Non-limiting examples of domains (e.g., transcription activator domains) of a synthetic transcription factor include ZF9, VP64, Rta, p65, and Hsf1 domains, either alone or combination. In some embodiments, a synthetic transcription factor may be a ZF9-VP64 fusion (e.g., VP64-Rta-p65 (VPR) fusion).

### Polynucleotides

In some embodiments, the present disclosure provides engineered polynucleotides. Engineered nucleic acids are not naturally occurring and may be produced recombinantly or synethtically. In some embodiments, the first and/or second polynucleotide is present on an expression vector, optionally a DNA plasmid.

Cells, in some embodiments, express engineered polynucleotides to produce components of the sequence detector systems of the present disclosure including, for example, a catalytically-inactive RNA-guided nuclease and/or a TALE. A cell may be transfected with engineered polynucleotides by any means known to a person skilled in the art, including but not limited to non-viral methods (e.g., calcium phosphate, lipofection, branched organic compounds, electroporation, cell squeezing, sonoporation, optical transfection, impalefection, etc.) and viral methods (e.g., adenoviruses, adeno-associated viruses, lentiviruses, retroviruses, etc.).

In some embodiments, the present disclosure provides a pair of engineered polynucleotides, wherein the first polynucleotide of the pair encodes in the 5′ (amino terminal) to 3′ (carboxy terminal) direction a first polypeptide, an N-terminal fragment of an intein, and a first catalytically-inactive RNA-guided nuclease, and optionally a first gRNA engineered to bind to a first target sequence, and the second polynucleotide of the pair encodes in the 5′ to 3′ direction a second catalytically-inactive RNA-guided nuclease, a C-terminal fragment of the intein, and a second polypeptide, and optionally a second gRNA engineered to bind to a second target sequence adjacent to the first target sequence. Expression of this pair of engineered polynucleotides and binding of the catalytically-inactive RNA-guided nucleases to the target sequences promotes intein removal, and the first and second polypeptides can be released. If the first and the second polypeptides are fragments of the same polypeptide, fusion of the two fragments will occur upon intein removal, resulting in polypeptide reconstitution.

In some embodiments, the present disclosure provides a pair of engineered polynucleotides, wherein the first polynucleotide of the pair encodes in the 5′ to 3′ direction a first polypeptide, an N-terminal fragment of an intein, and a first TALE effector DNA-binding domain (TALE) engineered to bind to a first target sequence, and the second polynucleotide of the pair encodes in the 5′ to 3′ direction a second TALE engineered to bind to a second targets sequence adjacent to the first target sequence, a C-terminal fragment of the intein, and a second polypeptide. Expression of this pair of engineered polynucleotides and binding of the TALE to the target sequences promotes intein removal, and the first and second polypeptides can be released. If the first and the second polypeptides are fragments of the same polypeptide, fusion of the two fragments will occur upon intein removal, resulting in polypeptide reconstitution.

In some embodiments, when the first polypeptide and the second polypeptide are joined, they form a synthetic transcription factor capable of activating transcription of a gene encoding a reporter molecule or a toxic molecule.

In some embodiments, the first polypeptide is an N-terminal fragment of a toxic molecule, and the second polypeptide is a C-terminal fragment of the toxic molecule.

### Methods of Use

In some embodiments, the present disclosure provides a cell comprising: (a) a sequence detector system and (b) a genome comprising the first and second target sequences. In some embodiments, the present disclosure provides a cell comprising: (a) a pair of engineered polynucleotides and (b) a genome comprising the first and second target sequences.

A cell may be either in vitro or in vivo. A cell may be a eukaryotic (e.g., mammalian or plant) or prokaryotic (e.g., bacterial) cell. In some embodiments, a cell is a mammalian cell, optionally a human cell, a pig cell, a mouse cell, a rat cell, a non-human primate cell, a dog cell, or a cat cell. In some embodiments, a cell is a human cell, optionally a liver cell, a kidney cell, a heart cell, a brain cell, a nerve cell, a blood cell, a T cell, a B cell, a stomach cell, a small intestine cell, a large intestine cell, a rectal cell, a bone cell, a pancreatic cell, an eye cell, a skin cell, or a connective tissue cell.

In some embodiments, the first target sequence and the second target sequence are separated from each other by fewer than 25 nucleotides. In some embodiments, the first target sequence and the second target sequence are separated by 25 to 50 nucleotides. In some embodiments, the first target sequence and the second target sequence are separated by 10 to 25 nucleotides. In some embodiments, the first target sequence and the second target sequence are separated by 5 to 25 nucleotides. In some embodiments, the first target sequence and the second target sequence are separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. The number of nucleotides that separate the first target sequence and the second target sequence may affect the efficiency of the sequence detector system, with more nucleotides decreasing the efficiency.

In some embodiments, the cell is a live cancer cell, optionally, in vitro, in situ, or in vivo. In some embodiments, the cancer cell is a liver cancer cell, a kidney cancer cell, a heart cancer cell, a brain cancer cell, a nerve cancer cell, a blood cancer cell, a T cell cancer, a B cell cancer, a stomach cancer cell, a small intestine cancer cell, a large intestine cancer cell, a rectal cancer cell, a bone cancer cell, a pancreatic cancer cell, an eye cancer cell, a skin cancer cell, or a connective tissue cancer cell.

In some embodiments, the first and second target sequences are cancer-specific target sequences. A cancer-specific target sequence is associated with or enriched in cancer cells compared with non-cancer cells. A cancer-associated sequence may be a deletion, an insertion, an expansion, a translocation, or a mutation in one or more residues in genes. Genes with deletion associated with cancer include tumor suppressor proteins (e.g., p53, RBP, Mdm2, PTEN, p16, WT1) and oncogene proteins (e.g., KLF6, EGFR, BRAF, BRCA1, and BRCA2). Genes with insertions associated with cancer include EGFR, HER2, KRAS, and MLL3. Genes with translocations associated with cancer include BCR and ABL (BCR-ABL fusion). Genes with mutations associated with cancer include, but are not limited to, BRCA1, BRCA2, p53, HER2, RAS.

In some embodiments, the present disclosure provides a selective detection method comprising delivering to a population of cells a pair of engineered polynucleotides and assaying for expression of activity of the reporter molecule. Selective detection refers to identifying cells expressing the reporter molecule. Assaying refers to analyzing (e.g., monitoring, measuring, observing) a population of cells for a reporter molecule. A population of cells may be in vitro, in situ, or in vivo.

In some embodiments, the present disclosure provides a selective ablation method comprising delivering to a population of cells a pair of engineered polynucleotides and assaying for cell death. Selective ablation refers to the death of cells that express a reporter molecule, wherein the reporter molecule is a toxin.

In some embodiments, the population of cells comprises cancer cells, and the first and second target sequences are specific to the cancer cells. In some embodiments, the cancer cells are in vitro, in situ, or in vivo. In some embodiments, the cancer cells are patient-derived. In some embodiments, the cancer cells are xenografts derived from patients and implanted into animals.

### Additional Embodiments

Additional embodiments of the present disclosure are encompassed by the following numbered paragraphs:

1. A sequence detector system comprising:
a first guide RNA (gRNA) and a first catalytically-inactive RNA-guided nuclease linked to an N-terminal fragment of an intein, wherein the N-terminal fragment is linked to a first polypeptide, and the first gRNA is engineered to bind to a first target sequence; and
a second gRNA and a second catalytically-inactive RNA-guided nuclease linked to an C-terminal fragment of an intein, wherein the C-terminal fragment is linked to a second polypeptide, and the second gRNA is engineered to bind to a second target sequence adjacent to the first target sequence,

wherein the first and second catalytically-inactive RNA-guided nucleases are orthogonal to each other.

2. The sequence detector system of paragraph 1, wherein the N-terminal fragment and the C-terminal fragment of the intein catalyze joining of the first polypeptide to the second polypeptide.

3. The sequence detector system of paragraph 1 or 2, wherein the first and second catalytically-inactive RNA-guided nucleases are selected from catalytically-inactive Cas nucleases and catalytically-inactive Cpf1 nucleases.

4. The sequence detector system of paragraph 3, wherein the first and second catalytically-inactive RNA-guided nucleases are selected from catalytically-inactive Streptococcus thermophiles Cas9 nuclease, Staphylococcus aureus Cas9 nucleases and Neisseria meningitidis Cas9 nucleases.

5. The sequence detector system of paragraph 4, wherein the first catalytically-inactive RNA-guided nuclease is a catalytically-inactive Streptococcus thermophiles Cas9 nuclease and the second catalytically-inactive RNA-guided nuclease is a catalytically-inactive Neisseria meningitidis Cas9 nuclease.

6. The sequence detector system of any one of paragraphs 1-5, wherein the intein is an engineered split intein or a naturally-occurring split intein.

7. The sequence detector system of paragraph 6, wherein the intein is selected from Saccharomyces cerevisiae VMA (Sce VMA) split inteins, Synechocystis sp. DnaB (Ssp DnaB) split inteins, Synechocystis sp. GyrB (Ssp GyrB) split inteins, Synechocystis sp. DnaE (Ssp DnaE) split inteins, and Nostoc punctiforme DnaE (Npu DnaE) split inteins.

8. The sequence detector system of any one of paragraphs 1-7, wherein

(a) the first polypeptide is a first reporter molecule and the second polypeptide is a second reporter molecule; or

(b) the first polypeptide is an N-terminal fragment of a reporter molecule and the second polypeptide is a C-terminal fragment of the reporter molecule.

9. The sequence detector of paragraph 8, wherein the first and/or second reporter molecule of (a) and/or the reporter molecule of (b) is selected from TagCFP, mTagCFP2, Azurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOκ, mKO2, mOrange, mOrange2, mRaspberry, mCherry, mStrawberry, mScarlet, mTangerine, tdTomato, TagRFP, TagRFP-T, mApple, mRuby, mRuby2, mPlum, HcRed-Tandem, mKate2, mNeptune, NirFP, TagRFP657, IFP1.4 and iRFP.

10. The sequence detector of paragraph 8 or 9, wherein the first and second reporter molecules of (a) are different from each other.

11. The sequence detector system of any one of paragraphs 1-7, wherein the first polypeptide is an N-terminal fragment of a toxic molecule and the second polypeptide is a C-terminal fragment of the toxic molecule.

12. The sequence detector of paragraph 11, wherein the toxic molecule is selected from toxins, pro-apoptotic proteins, and prodrug metabolic enzymes

13. The sequence detector system of any one of paragraphs 1-7, wherein

the first polypeptide is a first molecule of a synthetic transcription factor and the second polypeptide is a second molecule of the synthetic transcription factor; or

the first polypeptide is an N-terminal fragment of a synthetic transcription factor and the second polypeptide is a C-terminal fragment of the synthetic transcription factor.

14. The sequence detector system of paragraph 13, wherein the synthetic transcription factor binds to and activates transcription of a nucleic acid encoding a reporter molecule or a toxic molecule.

15. The sequence detector system of paragraph 14, wherein the nucleic acid encoding a reporter molecule or a toxic molecule comprises a minimal promoter and a binding site to which the synthetic transcription factor binds.

16. The sequence detector system of any one of paragraphs 1-15,

wherein the N terminus of the first catalytically-inactive RNA-guided nuclease is linked to the C terminus of the N-terminal fragment of the intein, the N terminus of the N-terminal fragment of the intein is linked to the C terminus of the first polypeptide, the C terminus of the second catalytically-inactive RNA-guided nuclease is linked to the N terminus of the C-terminal fragment of the intein, and the C terminus of the C-terminal fragment of the intein is linked to the N terminus of the second polypeptide.

17. A pair of engineered polynucleotides, wherein
the first polynucleotide of the pair encodes in the 5′ to 3′ direction a first polypeptide, an N-terminal fragment of an intein, a first catalytically-inactive RNA-guided nuclease, and
the second polynucleotide of the pair encodes in the 5′ to 3′ direction a second catalytically-inactive RNA-guided nuclease, a C-terminal fragment of the intein, and a second polypeptide,

wherein the first and second catalytically-inactive RNA-guided nucleases are orthogonal to each other.

18. The pair of engineered polynucleotides of paragraph 17, wherein the first polynucleotide further encodes a first guide RNA (gRNA) engineered to bind to a first target sequence, and the second polynucleotide further encodes a second gRNA engineered to bind to a second target sequence adjacent to the first target sequence.

19. The pair of engineered polynucleotides of paragraph 17 or 18, wherein the first and/or second polynucleotide is present on an expression vector, optionally a DNA plasmid.

20. The pair of engineered polynucleotides of any one of paragraphs 17-19, wherein the N-terminal fragment and the C-terminal fragment of the intein catalyze joining of the first polypeptide to the second polypeptide.

21. The pair of engineered polynucleotides of any one of paragraphs 17-20, wherein the first and second catalytically-inactive RNA-guided nucleases are selected from catalytically-inactive Cas nucleases and catalytically-inactive Cpf1 nucleases.

22. The pair of engineered polynucleotides of paragraph 21, wherein the first and second catalytically-inactive RNA-guided nucleases are selected from catalytically-inactive Streptococcus thermophiles Cas9 nuclease, Staphylococcus aureus Cas9 nucleases and Neisseria meningitidis Cas9 nucleases.

23. The pair of engineered polynucleotides of paragraph 22, wherein the first catalytically-inactive RNA-guided nuclease is a catalytically-inactive Streptococcus thermophiles Cas9 nuclease and the second catalytically-inactive RNA-guided nuclease is a catalytically-inactive Neisseria meningitidis Cas9 nuclease.

24. The pair of engineered polynucleotides of any one of paragraphs 17-23, wherein the intein is an engineered split intein or a naturally-occurring split intein.

25. The pair of engineered polynucleotides of paragraph 24, wherein the intein is selected from Saccharomyces cerevisiae VMA (Sce VMA) split inteins, Synechocystis sp. DnaB (Ssp DnaB) split inteins, Synechocystis sp. GyrB (Ssp GyrB) split inteins, Synechocystis sp. DnaE (Ssp DnaE) split inteins, and Nostoc punctiforme DnaE (Npu DnaE) split inteins.

26. The pair of engineered polynucleotides of any one of paragraphs 17-25, wherein

(a) the first polypeptide is a first reporter molecule and the second polypeptide is a second reporter molecule; or

(b) the first polypeptide is an N-terminal fragment of a reporter molecule and the second polypeptide is a C-terminal fragment of the reporter molecule.

27. The pair of engineered polynucleotides paragraph 26, wherein the first and/or second reporter molecule of (a) and/or the reporter molecule of (b) is selected from TagCFP, mTagCFP2, Azurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOκ, mKO2, mOrange, mOrange2, mRaspberry, mCherry, mStrawberry, mScarlet, mTangerine, tdTomato, TagRFP, TagRFP-T, mApple, mRuby, mRuby2, mPlum, HcRed-Tandem, mKate2, mNeptune, NirFP, TagRFP657, IFP1.4 and iRFP.

28. The pair of engineered polynucleotides paragraph 26 or 27, wherein the first and second reporter molecules of (a) are different from each other.

29. The pair of engineered polynucleotides of any one of paragraphs 17-25, wherein the first polypeptide is an N-terminal fragment of a toxic molecule and the second polypeptide is a C-terminal fragment of the toxic molecule.

30. The pair of engineered polynucleotides of paragraph 29, wherein the toxic molecule is selected from toxins, pro-apoptotic proteins, and prodrug metabolic enzymes

31. The pair of engineered polynucleotides of any one of paragraphs 17-25, wherein

the first polypeptide is a first molecule of a synthetic transcription factor and the second polypeptide is a second molecule of the synthetic transcription factor; or

the first polypeptide is an N-terminal fragment of a synthetic transcription factor and the second polypeptide is a C-terminal fragment of the synthetic transcription factor.

32. The pair of engineered polynucleotides of paragraph 31, wherein the synthetic transcription factor binds to and activates transcription of a nucleic acid encoding a reporter molecule or a toxic molecule.

33. The pair of engineered polynucleotides of paragraph 32, wherein the nucleic acid encoding a reporter molecule or a toxic molecule comprises a minimal promoter and a binding site to which the synthetic transcription factor binds.

34. A sequence detector system comprising:
a first TAL effector DNA-binding domain (TALE) linked to an N-terminal fragment of an intein, wherein the N-terminal fragment is linked to a first polypeptide, and the first TALE is engineered to bind to a first target sequence; and
a second TALE linked to an C-terminal fragment of an intein, wherein the C-terminal fragment is linked to a second polypeptide, and the second TALE is engineered to bind to a second target sequence adjacent to the first target sequence.

35. The sequence detector system of paragraph 34, wherein the N-terminal fragment and the C-terminal fragment of the intein catalyze joining of the first polypeptide to the second polypeptide.

36. The sequence detector system of paragraph 34 or 35, wherein the intein is an engineered split intein or a naturally-occurring split intein.

37. The sequence detector system of paragraph 36, wherein the intein is selected from Saccharomyces cerevisiae VMA (Sce VMA) split inteins, Synechocystis sp. DnaB (Ssp DnaB) split inteins, Synechocystis sp. GyrB (Ssp GyrB) split inteins, Synechocystis sp. DnaE (Ssp DnaE) split inteins, and Nostoc punctiforme DnaE (Npu DnaE) split inteins.

38. The sequence detector system of any one of paragraphs 34-37, wherein

(a) the first polypeptide is a first reporter molecule and the second polypeptide is a second reporter molecule; or

(b) the first polypeptide is an N-terminal fragment of a reporter molecule and the second polypeptide is a C-terminal fragment of the reporter molecule.

39. The sequence detector of paragraph 38, wherein the first and/or second reporter molecule of (a) and/or the reporter molecule of (b) is selected from TagCFP, mTagCFP2, Azurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOκ, mKO2, mOrange, mOrange2, mRaspberry, mCherry, mStrawberry, mScarlet, mTangerine, tdTomato, TagRFP, TagRFP-T, mApple, mRuby, mRuby2, mPlum, HcRed-Tandem, mKate2, mNeptune, NirFP, TagRFP657, IFP1.4 and iRFP.

40. The sequence detector of paragraph 38 or 39, wherein the first and second reporter molecules of (a) are different from each other.

41. The sequence detector system of any one of paragraphs 34-37, wherein the first polypeptide is an N-terminal fragment of a toxic molecule and the second polypeptide is a C-terminal fragment of the toxic molecule.

42. The sequence detector of paragraph 41, wherein the toxic molecule is selected from toxins, pro-apoptotic proteins, and prodrug metabolic enzymes

43. The sequence detector system of any one of paragraphs 34-37, wherein

the first polypeptide is a first molecule of a synthetic transcription factor and the second polypeptide is a second molecule of the synthetic transcription factor; or

the first polypeptide is an N-terminal fragment of a synthetic transcription factor and the second polypeptide is a C-terminal fragment of the synthetic transcription factor.

44. The sequence detector system of paragraph 43, wherein the synthetic transcription factor binds to and activates transcription of a nucleic acid encoding a reporter molecule or a toxic molecule.

45. The sequence detector system of paragraph 44, wherein the nucleic acid encoding a reporter molecule or a toxic molecule comprises a minimal promoter and a binding site to which the synthetic transcription factor binds.

46. The sequence detector system of any one of paragraphs 34-45,

wherein the N terminus of the first catalytically-inactive RNA-guided nuclease is linked to the C terminus of the N-terminal fragment of the intein, the N terminus of the N-terminal fragment of the intein is linked to the C terminus of the first polypeptide, the C terminus of the second catalytically-inactive RNA-guided nuclease is linked to the N terminus of the C-terminal fragment of the intein, and the C terminus of the C-terminal fragment of the intein is linked to the N terminus of the second polypeptide.

47. A pair of engineered polynucleotides, wherein
the first polynucleotide of the pair encodes in the 5′ to 3′ direction a first polypeptide, an N-terminal fragment of an intein, and a first TAL effector DNA-binding domain (TALE) engineered to bind to a first target sequence, and
the second polynucleotide of the pair encodes in the 5′ to 3′ direction a second TALE engineered to bind to a second target sequence adjacent to the first target sequence, a C-terminal fragment of the intein, and a second polypeptide.

48. The pair of engineered polynucleotides of paragraph 47, wherein the first and/or second polynucleotide is present on an expression vector, optionally a DNA plasmid.

49. The pair of engineered polynucleotides of paragraph 47 or 48, wherein the N-terminal fragment and the C-terminal fragment of the intein catalyze joining of the first polypeptide to the second polypeptide.

50. The pair of engineered polynucleotides of any one of paragraphs 47-49, wherein the intein is an engineered split intein or a naturally-occurring split intein.

51. The pair of engineered polynucleotides of paragraph 50, wherein the intein is selected from Saccharomyces cerevisiae VMA (Sce VMA) split inteins, Synechocystis sp. DnaB (Ssp DnaB) split inteins, Synechocystis sp. GyrB (Ssp GyrB) split inteins, Synechocystis sp. DnaE (Ssp DnaE) split inteins, and Nostoc punctiforme DnaE (Npu DnaE) split inteins.

52. The pair of engineered polynucleotides of any one of paragraphs 47-51, wherein

(a) the first polypeptide is a first reporter molecule and the second polypeptide is a second reporter molecule; or

(b) the first polypeptide is an N-terminal fragment of a reporter molecule and the second polypeptide is a C-terminal fragment of the reporter molecule.

53. The pair of engineered polynucleotides paragraph 52, wherein the first and/or second reporter molecule of (a) and/or the reporter molecule of (b) is selected from TagCFP, mTagCFP2, Azurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOκ, mKO2, mOrange, mOrange2, mRaspberry, mCherry, mStrawberry, mScarlet, mTangerine, tdTomato, TagRFP, TagRFP-T, mApple, mRuby, mRuby2, mPlum, HcRed-Tandem, mKate2, mNeptune, NirFP, TagRFP657, IFP1.4 and iRFP.

54. The pair of engineered polynucleotides paragraph 52 or 53, wherein the first and second reporter molecules of (a) are different from each other.

55. The pair of engineered polynucleotides of any one of paragraphs 47-51, wherein the first polypeptide is an N-terminal fragment of a toxic molecule and the second polypeptide is a C-terminal fragment of the toxic molecule.

56. The pair of engineered polynucleotides of paragraph 55, wherein the toxic molecule is selected from toxins, pro-apoptotic proteins, and prodrug metabolic enzymes

57. The pair of engineered polynucleotides of any one of paragraphs 47-51, wherein

the first polypeptide is a first molecule of a synthetic transcription factor and the second polypeptide is a second molecule of the synthetic transcription factor; or

the first polypeptide is an N-terminal fragment of a synthetic transcription factor and the second polypeptide is a C-terminal fragment of the synthetic transcription factor.

58. The pair of engineered polynucleotides of paragraph 57, wherein the synthetic transcription factor binds to and activates transcription of a nucleic acid encoding a reporter molecule or a toxic molecule.

59. The pair of engineered polynucleotides of paragraph 58, wherein the nucleic acid encoding a reporter molecule or a toxic molecule comprises a minimal promoter and a binding site to which the synthetic transcription factor binds.

60. A cell comprising: (a) the sequence detector system of any one of paragraphs 1-16 or 34-46 and (b) a genome comprising the first and second target sequences.

61. A cell comprising: (a) the pair of engineered polynucleotides of any one of paragraphs 17-33 or 47-59 and (b) a genome comprising the first and second target sequences.

62. The cell of paragraph 60 or 61, wherein the first target sequence and the second target sequence are separated from each by fewer than 25 nucleotides.

63. The cell of any one of paragraphs 60-62, wherein the cell is a live cancer cell, optionally in vitro, in situ, or in vivo.

64. The cell of paragraph 63, wherein the first and second target sequences are cancer-specific target sequences.

65. A selective detection method comprising delivering to a population of cells the pair of engineered polynucleotides of any one of paragraphs 26-28, 32, 33, 52-54, 58, or 59, and assaying for expression or activity of the reporter molecule.

66. A selective cell ablation method comprising delivering to a population of cells the pair of engineered polynucleotides of any one of paragraphs 29, 30, 32, 33, 55, 56, 58, or 59, and assaying for cell death.

67. The method of paragraphs 65 or 66, wherein the population of cells comprises cancer cells, and wherein the first and second target sequences are specific to the cancer cells.

### EXAMPLES

The present disclosure is further illustrated by the following Examples. These Examples are provided to aid in the understanding of the disclosure, and should not be construed as a limitation thereof.

### Example 1

### Develop and Test DNA Sequence Sensors for Gene Fusion

Two-Color In Vivo and In Situ Imaging of Fusion Genes.

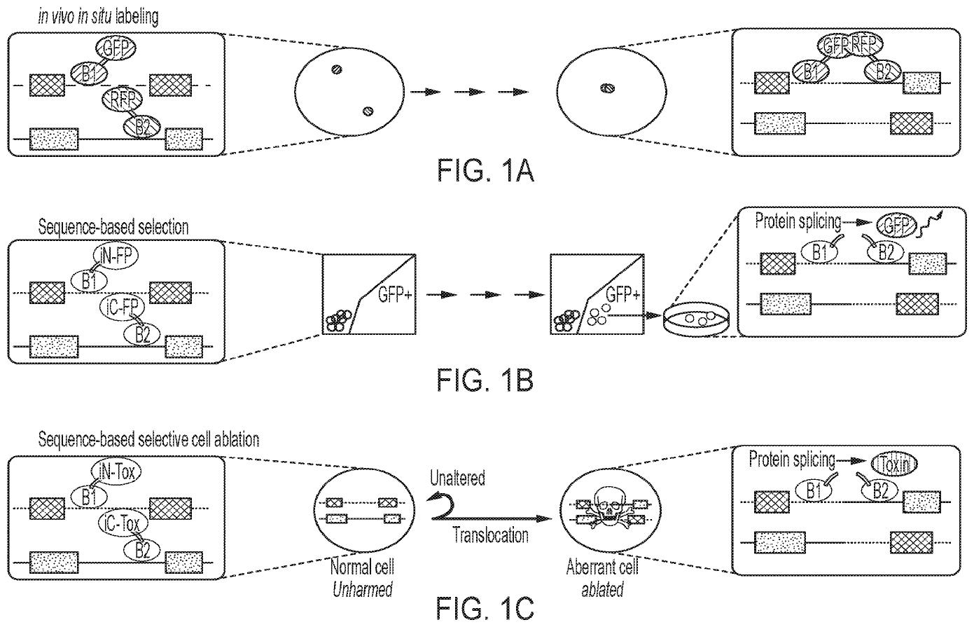

We first generate HEK293T cell lines with fusion genes EML4-ALK, CD74-ROS1 and AML1-ETO by CRISPR/Cas9 induced chromosomal translocation [13, 14]. Untreated HEK293T cells without fusion genes serve as the control. We transduce cells with lentiviruses expressing imaging components to label the 5′ junction with dCas9-GFP and the 3′ junction with dCas9-RFP for each translocation event in the translocation cell lines as well as wild-type HEK293T (FIG. 1A). Cells with unfused chromosomes (HEK293T-WT) will have disparate fluorescent foci while cells that have undergone the translocation event (e.g., HEK293T/EML4-ALK) will have a green focus overlapping with a red focus, resulting from the juxtaposition of the probes at the fusion junctions. We confirm specific labeling of junctions by DNA-FISH experiments.

Sequence-Based Selection of Cells Harboring Fusion Genes.

In this approach, a bipartite sensor, with each half tethering a non-functional signaling domain, reconstitutes functionality upon proximity-induced intein-mediated protein splicing [5] (FIG. 1B). Inteins are peptide elements from bacteria and yeast that can cleave themselves and join other parts of the protein together. Based on this feature, we use a DBD programmed to bind to the 5′ junctional sequence fused to N-terminal half of intein (iN) and to the N-terminal half of a marker (e.g., GFP) and another DBD programmed to bind to 3′ junctional sequence fused to C-terminal half of intein (iC) and to the other C-terminal half of a marker. Juxtaposition of the sensor halves through binding to a fusion sequence triggers protein splicing resulting in the joining of the GFP halves and the release of a full-length reconstituted GFP. Cells with the fused genome can thus be identified by fluorescent microscopy or fluorescence-activated cell sorting (FACS). With this technology, researchers can select live cells based on genotype in a high-throughput manner for downstream analysis. To facilitate the assessment of the specificity and sensitivity of these split probes in selecting for the cells containing the fusion genes, we first introduce a mCherry-expressing virus into the translocation cells (e.g., HEK293T/EML4-ALK), and introduce a TagBFP2-expressing virus into the HEK293T-WT cells. We mix the HEK293T-WT cells with the translocation cells, introduce the sensors into the cell mixture and then perform FACS analysis of the cells. To obtain a quantitative assessment, sensitivity is calculated by the % (GFP+mCherry+)/(mCherry+) while specificity is measured by % (GFP+mCherry+)/(GFP+). Sequence-based selection results in all mCherry+cells being GFP+, and vice versa, and TagBFP2+ and GFP are mutually exclusive.

Sequence-Based Selective Cell Ablation.

In this approach, a protein splicing strategy is used to reconstitute a toxin, or a pro-apoptotic protein, or a prodrug metabolic enzyme upon juxtaposition of the sensor halves via genome rearrangement (FIG. 1C). In normal cells, the sensors are separate and do not produce a functional toxin or apoptosis trigger. Cells containing fusion genes arising from genomic rearrangement events will contain the fusion sequences juxtaposing the sensor halves to reconstitute the toxin. Likewise, a prodrug metabolic enzyme can be reconstituted in cells with a fusion gene, while cells without fusion genes will not have such a conversion, sparing WT cells from the toxic effect of the metabolized drug. This technology may be used as a therapeutic strategy to kill cells upon genomic rearrangement to prevent them from propagating. To test the various devices for selective cell ablation, HEK293T-WT cells expressing TagBFP2 and the translocation cells (e.g., HEK293T/EML4-ALK) expressing mCherry are mixed together, then the cell mixture transduced with the ablation devices, or mock-transduced, and in the case of prodrug metabolic enzyme reconstitution, incubated with or without the prodrug. The cells are then be subjected to a time course of FACS experiments (e.g., Day 0, Day 1, Day 2, Day 3, Day 7, Day 14) to quantify the ratio of TagBFP2+ cells (HEK293T-WT) vs mCherry+ cells (translocation cells). An ideal selective cell ablation will deplete the mCherry+ cells. To quantify cell death, HEK293T-WT and translocation cells will be assayed independently for apoptosis assays, or growth curve with or without the ablation devices, with or without the drug if applicable.

### Example 2

### CRISPR/Cas9 Based Sequence Detectors (CRISPR.Sense)

Catalytically-inactive Cas9 (dCas9) proteins act as RNA-guided DNA binding proteins that are easily programmed to bind without cutting target DNA sequence. The specificity is determined by a guide RNA containing a sequence that matches the targeted sites. An engineered dCas9 sequence detector pair can serve any targeted sequence by providing specific guide RNA without de novo generation of sequence detector modules for each sequence target.

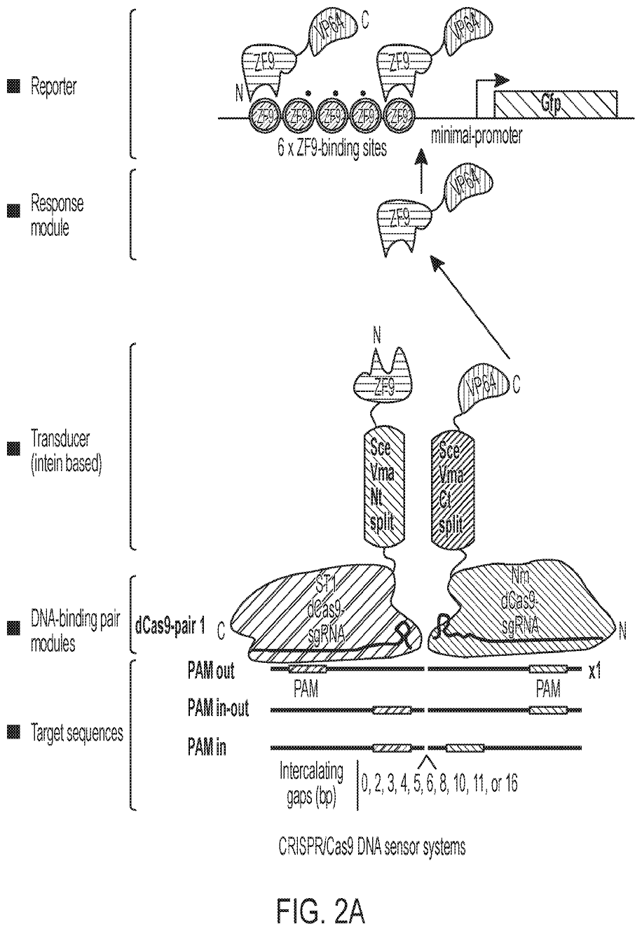

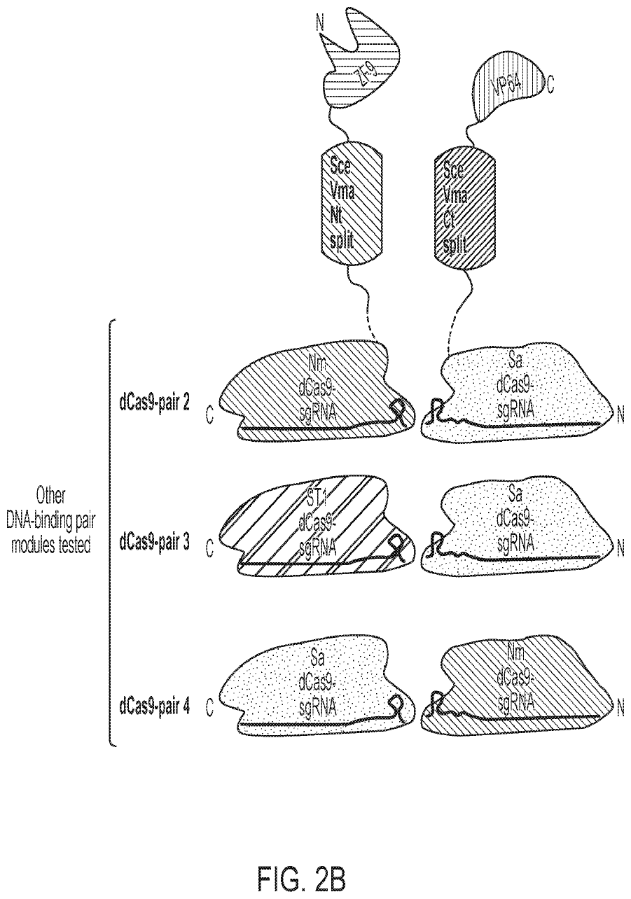

The bipartite nature of the target sites uses independent programming of the dCas9 DNA-binding modules. Orthogonal dCas9 proteins can be used as DNA-binding pair modules as their respective sgRNAs are species specific. dCas9 of Streptococcus thermophilus (ST1 dCas9), Staphylococcus aureus (Sa dCas9) and Neisseria meningitidis (Nm dCas9) and their respective guide RNAs were used to construct four pairs of dCas9-based sequence detectors (FIGS. 2A-2B) [6, 7].

To allow probing for optimal configuration and spacing required for efficient binding of the two dCas9 partners of each sensor, synthetic template targets that comprised sequences that matched the corresponding sgRNA and protospacer adjacent motif (PAM) sequences required for target recognition in all possible configurations were made (PAM in”, “PAM out”, or “PAM in-out”) (FIG. 2A). The sequence targets of the bipartite binding sites were separated by a gap of various length (FIG. 2A). The sequence targets were selected based on screens for guide RNAs that efficiently enabled the respective CRISPR/Cas9-mediated cleavage within the tdTomato coding sequence of a HEK293T derived cell line.

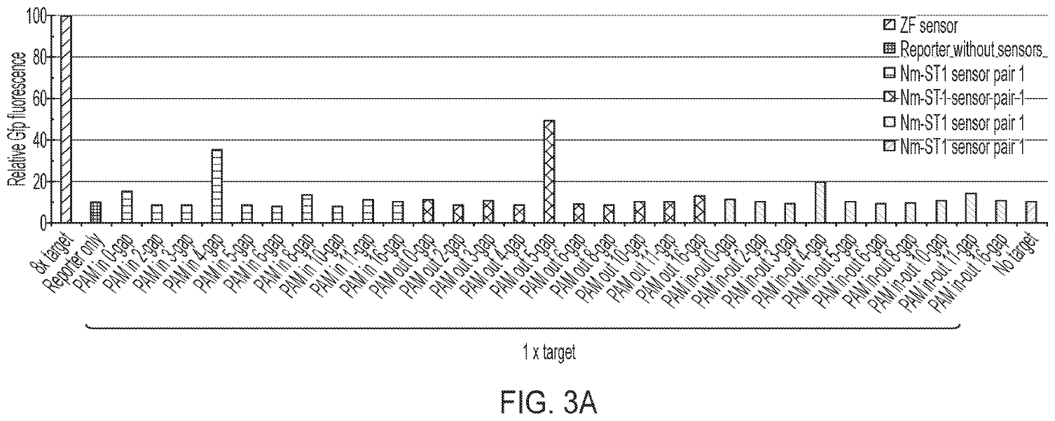

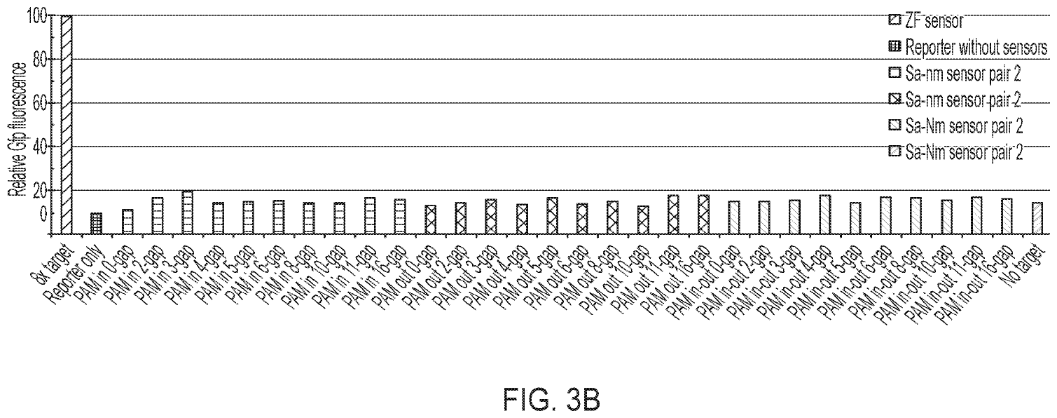

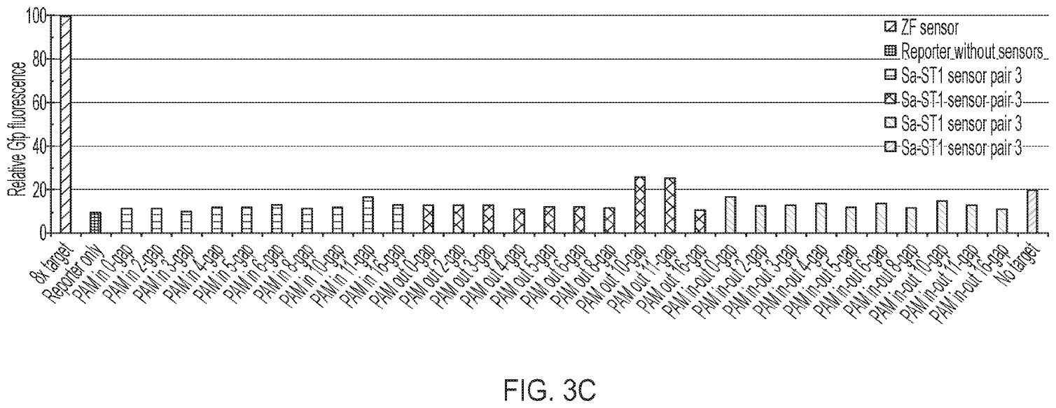

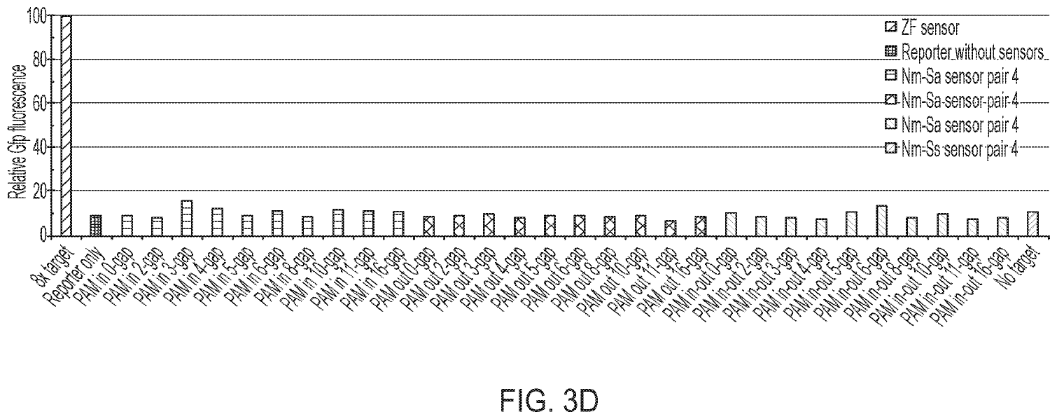

To determine the efficiency of dCas9-based sequence detectors, each of the pairs were compared to a ZF DNA sensor system using the GFP-based reporter and the replicative plasmid containing 8 copies of the target sequences [1]. For the dCas9-based sequence detector pairs, a single copy of a synthetic sequence target replaced the sequence targets of the ZF-based sequence detector within the replicative plasmid. Transfection of HEK293T cells with plasmid components of each system and FACS analyses showed that 40 to 50% activity relative to the ZF DNA sensor system was obtained with the Nm-ST1 dCas9 sensor paid when the target sequences contained 4 or 5 bp gap and PAM sequences in “PAM in” or “PAM out” configuration respectively (FIG. 3A). This is indeed of significance as the dCas9-sequence detector systems were tested on plasmids comprising one copy of the target sequence whereas eight copies of the target sequence were used with the ZF-based system The presence of multiple targets presumably allows amplification of the response as more binding events result in higher frequency of trans-splicing the reporter module and GFP synthesis. Further optimization of Nm-ST1 sequence detector pair 1 holds promises for a greater efficiency of detection.

Unexpectedly, the dCas9 sequence detector pairs 2, 3, and 4 did not work with all the tested target sequences as indicated by the obtained background GFP levels (FIGS. 3B-3D). The failure of the dCas9 sequence detector pairs 2, 3, and 4 could be due to several factors, further experiments are needed to establish conditions for these to work.

### Example 3

### TALE-Based Sequence Detectors (TALE.Sense)

To test the sensitivity of sequence detector, the dCas9 proteins were replaced with the transcription activator-like effector (TALE) modules of Xanthomonas sp. [3, 4]. Advances in programming DNA binding proteins using TALE modules allows convenient assembly of highly specific DNA-binding proteins [5]. Each TALE module recognizes a single base-pair (bp) (as opposed to a triplet bp for ZF modules), making the TALE modules assembly straightforward.

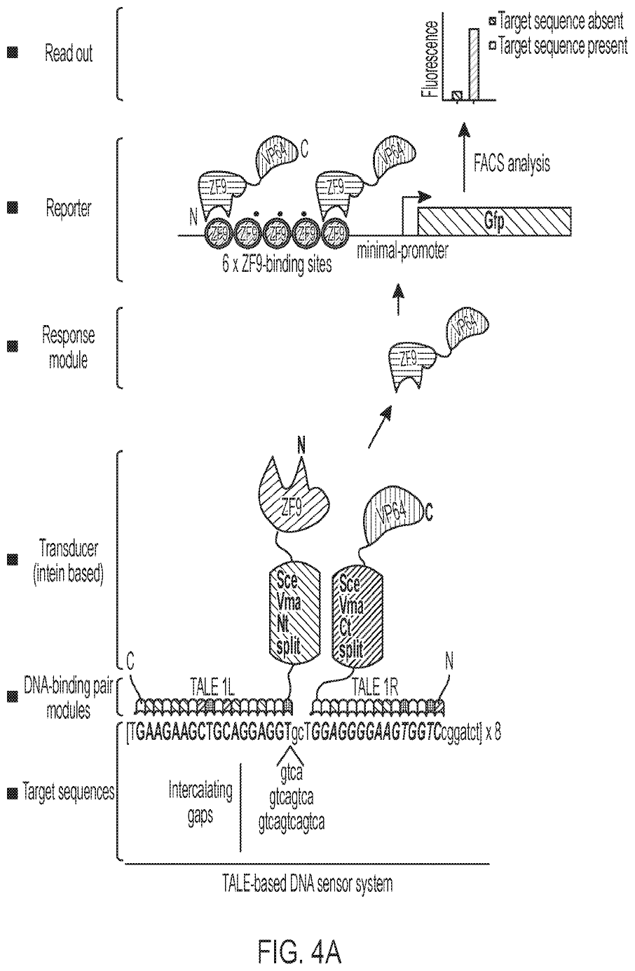

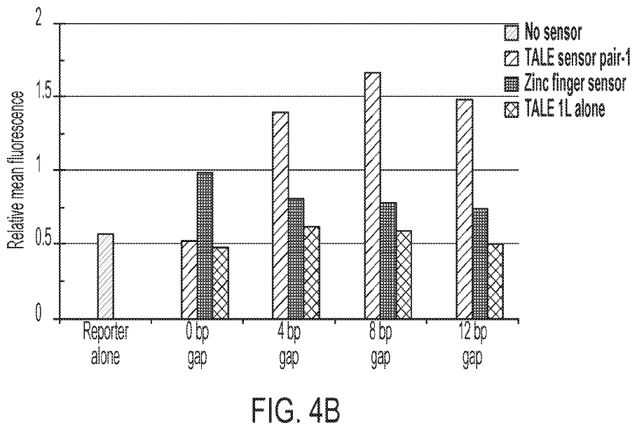

To assess a TALE-based sequence detector, a TALE pair (TALE pair-1) programmed to bind to the same target sequences of a ZF-based DNA sensor was assembled (FIG. 4A left side) [1]. The TALE sequence detector and ZF-based DNA sensor were therefore tested against previously reported non-replicative plasmids containing 8 copies of target sequences with varying lengths of the gaps separating the sensor's target sites (0, 4, 8, 12 bps). Transfection of HEK293T cells with plasmids components of the systems and fluorescence-activated cell sorting (FACS) analysis 72 h after transfection showed that TALE sequence detector-1 gave higher activity over a wide range of target sequences containing 4, 8, 12 bp gaps separating the binding sites (FIG. 4B). Consistent with earlier report, the ZF-sensor was most active when no gap existed between target sites and the activity was reduced when gaps were present (FIG. 4B) [1].The activity obtained with the TALE sequence detector requires expression of both TALE DNA-binding pair as transfection with a single partner showed the basal GFP level obtained when cells were transfected with the reporter plasmid only (FIG. 4B).

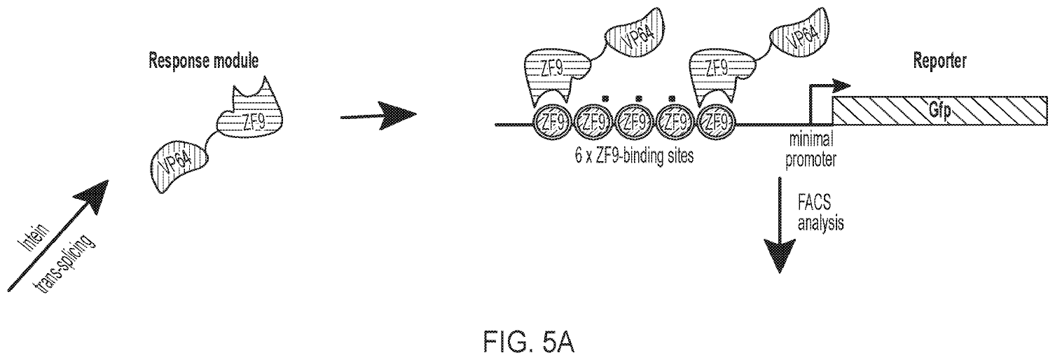

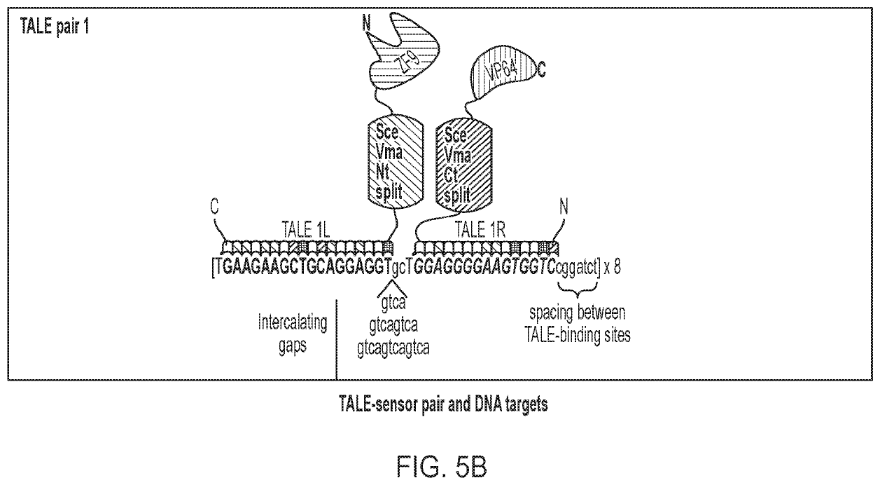

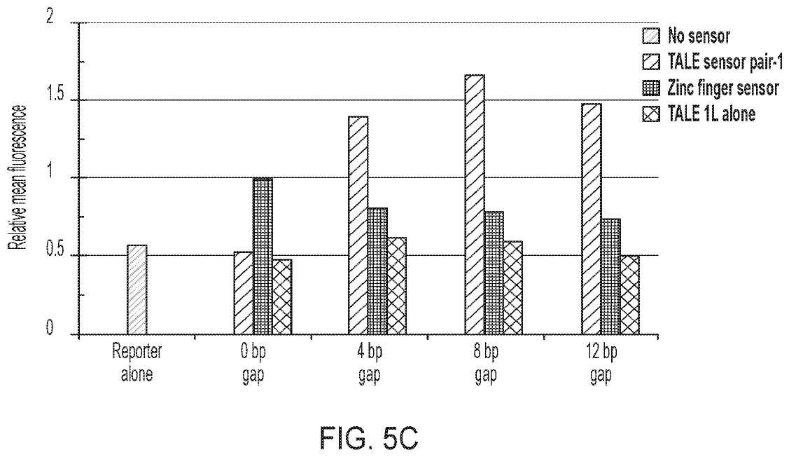

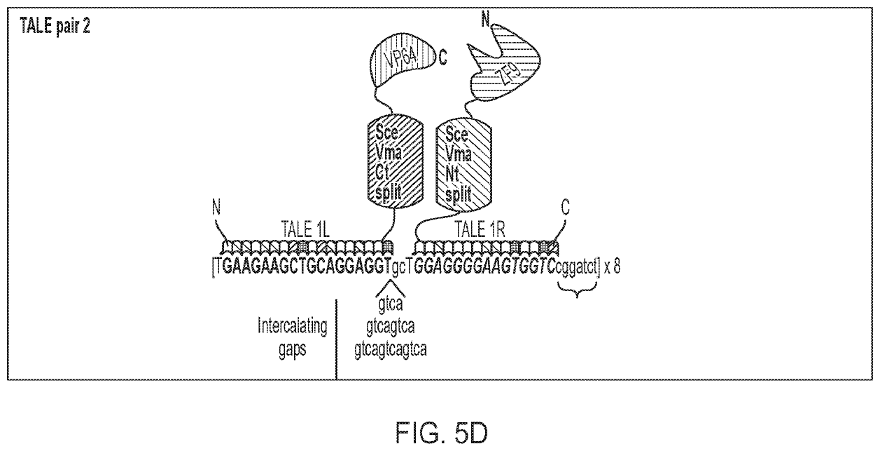

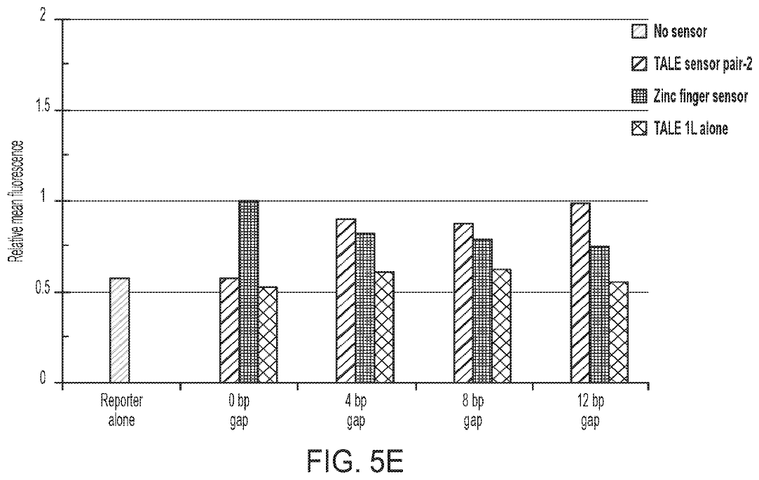

To further determine the structural requirements for the TALE-based sequence detectors the sensor pair-1 was altered by swapping the Ct-intein split-VP64 and Nt-intein split-ZF9 fusion within the sensor pair (FIG. 5B, FIG. 5D). The obtained TALE sensor pair-2 was then compared to the ZF DNA sensor sequence detector by using previously reported non-replicative plasmids containing 8 copies of target sequences with varying lengths of the gaps separating the sensor's target sites (0, 4, 8, 12 bps). This showed a slightly higher activity with 4-12 bp gaps than the ZF-based DNA, however the overall activity was much lower that obtained with the TALE sequence detector-1 (FIG. 5C, FIG. 5E). This indicates the existence of topological requirements for an efficient intein trans-splicing and/or binding target sequences. It appears that the TALE sequence detectors are more effective when the ZF9-Nt intein fusion is associated with the TALE sequence detector arm that binds the left side of the target site, and the Ct-intein-VP64 is linked to the TALE sequence detector partner that binds the opposite side of the target sites.

Taken together the data indicate that the use of TALE domains simplifies the engineering of sequence detectors and also enables efficient detection of a broad range of target sequences. Thus, this sequence detector platform is a versatile DNA sensing tool for numerous applications.

### Example 4

### TALE Sequences Detector Detects Non-Repeat DNA Sequences

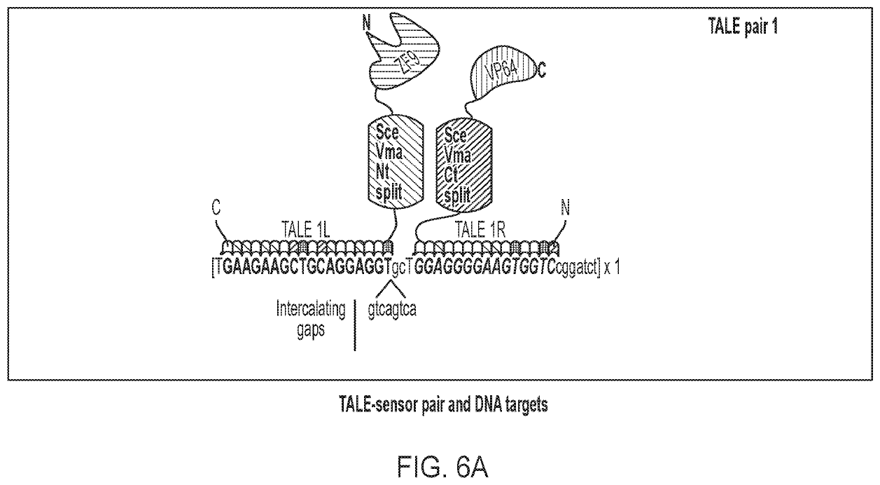

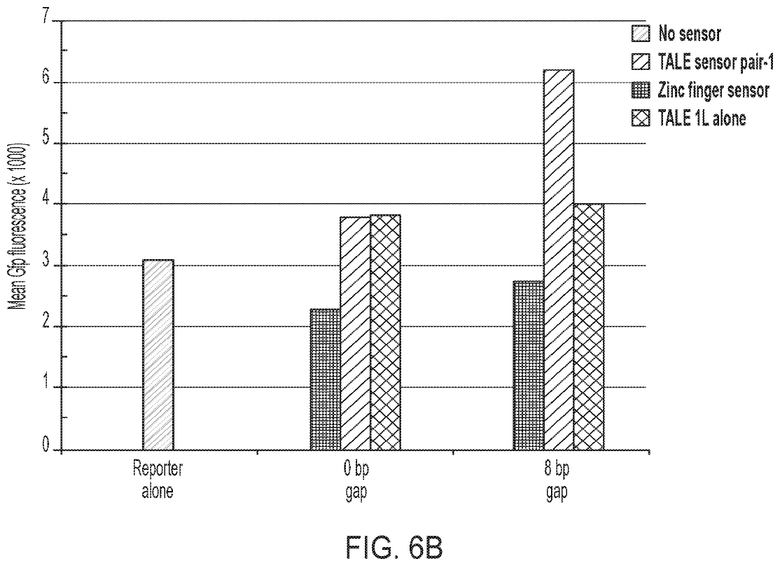

A sequence detector system would be of a greater significance if it enables detection of non-repeated DNA sequences as those present on many chromosomes either as native sequences or result from changes upon genome editing, viral infections or aberrant chromosomal rearrangements. The TALE sequence detector-1 and the ZF DNA sensor were compared in their ability to report the presence of a target sequence present as single copy within a non-replicative plasmid. This showed that the ZF DNA sensor failed to sense and report on all the tested targets including the one with optimal gap size as indicated by the obtained background levels of GFP (FIG. 6B). In contrast, the TALE sequence detector-1 induced a significant activity with 8 bp gap-target substrate (FIG. 6A, FIG. 6B). The obtained activity with TALE sequence detector-1 required the presence both DNA-binding partners of the system (TALE 1L and TALE 1R) as only background levels were obtained when cells were transfected with TALE 1L partner alone (FIG. 6B).

Taken together the data show that the TALE-based sequence detector developed herein is more sensitive and efficient compared to the ZF based DNA sensor [1]. The TALE-based sequence detector may be used for identifying, isolating, or targeting a subset of cellular variants harboring for example viral sequences or DNA sequences that emerged from chromosomal rearrangements found in certain cancer cell types, for example.

The GFP in the reporter could be replaced by, for example, an enzyme that converts an inert substrate to a cytotoxic drug and therefore allows elimination of cells that contain targeted DNA sequences. With its high efficiency and sensitivity, the TALE.Sense technology hold promises for developing novel therapies.

### Materials and Methods

Cell Culture and Transfection

HEK293T cells were cultivated in Dulbecco's modified Eagle's medium (DMEM)(Sigma) with 10% fetal bovine serum (FBS)(Lonza), 4% Glutamax (Gibco), 1% Sodium Pyruvate (Gibco) and penicillin-streptomycin (Gibco) in an incubator set to 37° C. and 5% CO2. Cells were seeded into 96-well plates at 30,000 cells per well the day before being transfected with a 400 ng plasmid DNA using Attractene transfection reagent according to manufacturer's instructions (Qiagen). Plasmid DNA mixes used to transfect cells contained a reporter, target, and sensor expression plasmids at 1:1:1 mass ratio of respectively. Cells were harvested 48 or 72 hours after transfection and analyzed by FACS.

Fluorescence-Activated Cell Sorting

Cells were detached from plate by treatment with 0.05% of Trypsin: EDTA for 5 min at 37 C and then suspended in the culture medium. Samples were analyzed on a LSRFortessa X-20 flow cytometer using a high-throughput plate sampler and FACSDiVA 8.0 software (BD Bioscience). Five thousand events were collected in each run.

### Constructs and Sequences

TABLE 1
List of plasmids

| | | Addgene |
|---|---|---|
| Plasmid ID | Description | ID |
| pLH-nmsgRNA1.1 | U6 promoter for Nm-sgRNA expression | 64115 |
| pLH-St sgRNA2.1 | U6 promoter for ST1-sgRNA expression | 64117 |
| pAT399 | U6 promoter for Sa-sgRNA | pending |
| | expression. Derived from | |
| | Addgene plasmid # 61591 by | |
| | removing Cas9 coding | |
| | sequence | |
| pVITRO1_SS_269 | ZF sensor expression | 68771 |
| pGL4.26-SS-192 | GFP reporter | 68759 |
| pBW121-SS-315 | Replicative plasmid 8x | 68786 |
| | target sites with no gap | |
| pBW121-SS-309 | 8x target sites with no gap | 68777 |
| pBW121-SS-310 | 8x target sites with 4 bp gap | 68778 |
| pBW121-SS-287 | 8x target sites with 8 bp gap | 68779 |
| pBW121-SS-311 | 8x target sites with 12 bp gap | 68780 |
| pBW121-SS-289 | lx target site with no gap | 68781 |
| pBW121-SS-287- | lx target site with 8 bp gap | pending |
| AT | (derivative of pBW121-SS-287) | |
| pAT643 | TALE sensor pair 2 | pending |
| pAT644 | TALE sensor pair 1 | pending |
| pAT1 | dCas9 sensor pair 2 | pending |
| pAT2 | dCas9 sensor pair 3 | pending |
| pAT3 | dCas9 sensor pair 4 | pending |
| pAT4 | dCas9 sensor pair 1 | pending |

TABLE 2
Spacer sequences of sgRNAs targeting tdTomato
gene

| | | | SEQ ID |
|---|---|---|---|
| sgRNA | spacer sequences | Reference | NO: |
| sgNm-1 | TACGTGAAGCACCCCGCCGACA | [8] | 1 |
| | T | | |
| sgSa-6 | TTCTTGTAATCGGGGATGTCG | This work | 2 |
| sgST1-10 | CCCGCCGACATCCCCGATTA | This work | 3 |

TABLE 3
Sequence of target sites for CRISPR.sense in pBW121-SS-315

| Gap | | SEQ ID |
|---|---|---|
| (bp) | Nm-ST1 “PAM in” target sequence | NO: |
| 0 | TACGTGAAGCACCCCGCCGACATccccGATTTTCTtgTAATCGGG | 4 |
| | GATGTCGGCGGG | |
| 2 | TACGTGAAGCACCCCGCCGACATccccGATTacTTCTtgTAATCG | 5 |
| | GGGATGTCGGCGGG | |
| 3 | TACGTGAAGCACCCCGCCGACATccccGATTacgTTCTtgTAATCG | 6 |
| | GGGATGTCGGCGGG | |
| 4 | TACGTGAAGCACCCCGCCGACATccccGATTactgTTCTtgTAATC | 7 |
| | GGGGATGTCGGCGGG | |
| 5 | TACGTGAAGCACCCCGCCGACATccccGATTactgaTTCTtgTAATC | 8 |
| | GGGGATGTCGGCGGG | |
| 6 | TACGTGAAGCACCCCGCCGACATccccGATTactgacTTCTtgTAAT | 9 |
| | CGGGGATGTCGGCGGG | |
| 8 | TACGTGAAGCACCCCGCCGACATccccGATTactgactgTTCTtgTAA | 10 |
| | TCGGGGATGTCGGCGGG | |
| 10 | TACGTGAAGCACCCCGCCGACATccccGATTactgactgacTTCTtgTA | 11 |
| | ATCGGGGATGTCGGCGGG | |
| 11 | TACGTGAAGCACCCCGCCGACATccccGATTactgactgacgTTCTtgT | 12 |
| | AATCGGGGATGTCGGCGGG | |
| 16 | TACGTGAAGCACCCCGCCGACATccccGATTactgactgactgactgTTCT | 13 |
| | tgTAATCGGGGATGTCGGCGGG | |
| Gap | Nm-ST1 “PAM out” target sequence | 14 |
| 0 | AATCggggATGTCGGCGGGGTGCTTCACGTACCCGCCGACATC | 15 |
| | CCCGATTAcaAGAA | |
| 2 | AATCggggATGTCGGCGGGGTGCTTCACGTAacCCCGCCGACAT | 16 |
| | CCCCGATTAcaAGAA | |
| 3 | AATCggggATGTCGGCGGGGTGCTTCACGTAacgCCCGCCGACA | 17 |
| | TCCCCGATTAcaAGAA | |
| 4 | AATCggggATGTCGGCGGGGTGCTTCACGTAactgCCCGCCGACA | 18 |
| | TCCCCGATTAcaAGAA | |
| 5 | AATCggggATGTCGGCGGGGTGCTTCACGTAactgaCCCGCCGAC | 19 |
| | ATCCCCGATTAcaAGAA | |
| 6 | AATCggggATGTCGGCGGGGTGCTTCACGTAactgacCCCGCCGAC | 20 |
| | ATCCCCGATTAcaAGAA | |
| 8 | AATCggggATGTCGGCGGGGTGCTTCACGTAactgactgCCCGCCGA | 21 |
| | CATCCCCGATTAcaAGAA | |
| 10 | AATCggggATGTCGGCGGGGTGCTTCACGTAactgactgacCCCGCC | 22 |
| | GACATCCCCGATTAcaAGAA | |
| 11 | AATCggggATGTCGGCGGGGTGCTTCACGTAactgactgacgCCCGCC | 23 |
| | GACATCCCCGATTAcaAGAA | |
| 16 | AATCggggATGTCGGCGGGGTGCTTCACGTAactgactgactgactgCCC | 24 |
| | GCCGACATCCCCGATTAcaAGAA | |
| Gap | Nm-ST1 “PAM in-out” target sequence | 25 |
| 0 | TACGTGAAGCACCCCGCCGACATccccGATTCCCGCCGACATC | 26 |
| | CCCGATTAcaAGAA | |
| 2 | TACGTGAAGCACCCCGCCGACATccccGATTacCCCGCCGACAT | 27 |
| | CCCCGATTAcaAGAA | |
| 3 | TACGTGAAGCACCCCGCCGACATccccGATTacgCCCGCCGACAT | 28 |
| | CCCCGATTAcaAGAA | |
| 4 | TACGTGAAGCACCCCGCCGACATccccGATTactgCCCGCCGACA | 29 |
| | TCCCCGATTAcaAGAA | |
| 5 | TACGTGAAGCACCCCGCCGACATccccGATTactgaCCCGCCGAC | 30 |
| | ATCCCCGATTAcaAGAA | |
| 6 | TACGTGAAGCACCCCGCCGACATccccGATTactgacCCCGCCGAC | 31 |
| | ATCCCCGATTAcaAGAA | |
| 8 | TACGTGAAGCACCCCGCCGACATccccGATTactgactgCCCGCCGA | 32 |
| | CATCCCCGATTAcaAGAA | |
| 10 | TACGTGAAGCACCCCGCCGACATccccGATTactgactgacCCCGCCG | 33 |
| | ACATCCCCGATTAcaAGAA | |
| 11 | TACGTGAAGCACCCCGCCGACATccccGATTactgactgacgCCCGCC | 34 |
| | GACATCCCCGATTAcaAGAA | |
| 16 | TACGTGAAGCACCCCGCCGACATccccGATTactgactgactgactgCCC | 35 |
| | GCCGACATCCCCGATTAcaAGAA | |
| Gap | Sa-Nm “PAM in” target sequence | 36 |
| 0 | TTCTTGTAATCGGGGATGTCGGcgGGGTAATCggggATGTCGGC | 37 |
| | GGGGTGCTTCACGTA | |
| 2 | TTCTTGTAATCGGGGATGTCGGcgGGGTacAATCggggATGTCGG | 38 |
| | CGGGGTGCTTCACGTA | |
| 3 | TTCTTGTAATCGGGGATGTCGGcgGGGTacgAATCggggATGTCG | 39 |
| | GCGGGGTGCTTCACGTA | |
| 4 | TTCTTGTAATCGGGGATGTCGGcgGGGTactgAATCggggATGTCG | 40 |
| | GCGGGGTGCTTCACGTA | |
| 5 | TTCTTGTAATCGGGGATGTCGGcgGGGTactgaAATCggggATGTC | 41 |
| | GGCGGGGTGCTTCACGTA | |
| 6 | TTCTTGTAATCGGGGATGTCGGcgGGGTactgacAATCggggATGTC | 42 |
| | GGCGGGGTGCTTCACGTA | |
| 8 | TTCTTGTAATCGGGGATGTCGGcgGGGTactgactgAATCggggATGT | 43 |
| | CGGCGGGGTGCTTCACGTA | |
| 10 | TTCTTGTAATCGGGGATGTCGGcgGGGTactgactgacAATCggggAT | 44 |
| | GTCGGCGGGGTGCTTCACGTA | |
| 11 | TTCTTGTAATCGGGGATGTCGGcgGGGTactgactgacgAATCggggAT | 45 |
| | GTCGGCGGGGTGCTTCACGTA | |
| 16 | TTCTTGTAATCGGGGATGTCGGcgGGGTactgactgactgactgAATCggg | 46 |
| | gATGTCGGCGGGGTGCTTCACGTA | |
| Gap | Sa-Nm “PAM out”·target sequence | 47 |
| 0 | ACCCcgCCGACATCCCCGATTACAAGAATACGTGAAGCACCC | 48 |
| | CGCCGACATccccGATT | |
| 2 | ACCCcgCCGACATCCCCGATTACAAGAAacTACGTGAAGCACC | 49 |
| | CCGCCGACATccccGATT | |
| 3 | ACCCcgCCGACATCCCCGATTACAAGAAacgTACGTGAAGCACC | 50 |
| | CCGCCGACATccccGATT | |
| 4 | ACCCcgCCGACATCCCCGATTACAAGAAactgTACGTGAAGCAC | 51 |
| | CCCGCCGACATccccGATT | |
| 5 | ACCCcgCCGACATCCCCGATTACAAGAAactgaTACGTGAAGCAC | 52 |
| | CCCGCCGACATccccGATT | |
| 6 | ACCCcgCCGACATCCCCGATTACAAGAAactgacTACGTGAAGCA | 53 |
| | CCCCGCCGACATccccGATT | |
| 8 | ACCCcgCCGACATCCCCGATTACAAGAAactgactgTACGTGAAGC | 54 |
| | ACCCCGCCGACATccccGATT | |
| 10 | ACCCcgCCGACATCCCCGATTACAAGAAactgactgacTACGTGAAG | 55 |
| | CACCCCGCCGACATccccGATT | |
| 11 | ACCCcgCCGACATCCCCGATTACAAGAAactgactgacgTACGTGAA | 56 |
| | GCACCCCGCCGACATccccGATT | |
| 16 | ACCCcgCCGACATCCCCGATTACAAGAAactgactgactgactgTACGT | 57 |
| | GAAGCACCCCGCCGACATccccGATT | |
| Gap | Sa-Nm “PAM in-out” target sequence | 58 |
| 0 | TTCTTGTAATCGGGGATGTCGGcgGGGTTACGTGAAGCACCCC | 59 |
| | GCCGACATccccGATT | |
| 2 | TTCTTGTAATCGGGGATGTCGGcgGGGTacTACGTGAAGCACCC | 60 |
| | CGCCGACATccccGATT | |
| 3 | TTCTTGTAATCGGGGATGTCGGcgGGGTacgTACGTGAAGCACC | 61 |
| | CCGCCGACATccccGATT | |
| 4 | TTCTTGTAATCGGGGATGTCGGcgGGGTactgTACGTGAAGCAC | 62 |
| | CCCGCCGACATccccGATT | |
| 5 | TTCTTGTAATCGGGGATGTCGGcgGGGTactgaTACGTGAAGCAC | 63 |
| | CCCGCCGACATccccGATT | |
| 6 | TTCTTGTAATCGGGGATGTCGGcgGGGTactgacTACGTGAAGCA | 64 |
| | CCCCGCCGACATccccGATT | |
| 8 | TTCTTGTAATCGGGGATGTCGGcgGGGTactgactgTACGTGAAGC | 65 |
| | ACCCCGCCGACATccccGATT | |
| 10 | TTCTTGTAATCGGGGATGTCGGcgGGGTactgactgacTACGTGAAG | 66 |
| | CACCCCGCCGACATccccGATT | |
| 11 | TTCTTGTAATCGGGGATGTCGGcgGGGTactgactgacgTACGTGAA | 67 |
| | GCACCCCGCCGACATccccGATT | |
| 16 | TTCTTGTAATCGGGGATGTCGGcgGGGTactgactgactgactgTACGTG | 68 |
| | AAGCACCCCGCCGACATccccGATT | |
| Gap | Sa-ST1 “PAM in” target sequence | 69 |
| 0 | TTCTTGTAATCGGGGATGTCGGcgGGGTTTCTtgTAATCGGGGA | 70 |
| | TGTCGGCGGG | |
| 2 | TTCTTGTAATCGGGGATGTCGGcgGGGTacTTCTtgTAATCGGGG | 71 |
| | ATGTCGGCGGG | |
| 3 | TTCTTGTAATCGGGGATGTCGGcgGGGTacgTTCTtgTAATCGGG | 72 |
| | GATGTCGGCGGG | |
| 4 | TTCTTGTAATCGGGGATGTCGGcgGGGTactgTTCTtgTAATCGGG | 73 |
| | GATGTCGGCGGG | |
| 5 | TTCTTGTAATCGGGGATGTCGGcgGGGTactgaTTCTtgTAATCGG | 74 |
| | GGATGTCGGCGGG | |
| 6 | TTCTTGTAATCGGGGATGTCGGcgGGGTactgacTTCTtgTAATCG | 75 |
| | GGGATGTCGGCGGG | |
| 8 | TTCTTGTAATCGGGGATGTCGGcgGGGTactgactgTTCTtgTAATC | 76 |
| | GGGGATGTCGGCGGG | |
| 10 | TTCTTGTAATCGGGGATGTCGGcgGGGTactgactgacTTCTtgTAAT | 77 |
| | CGGGGATGTCGGCGGG | |
| 11 | TTCTTGTAATCGGGGATGTCGGcgGGGTactgactgacgTTCTtgTAAT | 78 |
| | CGGGGATGTCGGCGGG | |
| 16 | TTCTTGTAATCGGGGATGTCGGcgGGGTactgactgactgactgTTCTtgT | 79 |
| | AATCGGGGATGTCGGCGGG | |
| Gap | Sa-ST1 “PAM out” target sequence | 80 |
| 0 | ACCCcgCCGACATCCCCGATTACAAGAACCCGCCGACATCCC | 81 |
| | CGATTAcaAGAA | |
| 2 | ACCCcgCCGACATCCCCGATTACAAGAAacCCCGCCGACATCC | 82 |
| | CCGATTAcaAGAA | |
| 3 | ACCCcgCCGACATCCCCGATTACAAGAAacgCCCGCCGACATCC | 83 |
| | CCGATTAcaAGAA | |
| 4 | ACCCcgCCGACATCCCCGATTACAAGAAactgCCCGCCGACATC | 84 |
| | CCCGATTAcaAGAA | |
| 5 | ACCCcgCCGACATCCCCGATTACAAGAAactgaCCCGCCGACATC | 85 |
| | CCCGATTAcaAGAA | |
| 6 | ACCCcgCCGACATCCCCGATTACAAGAAactgacCCCGCCGACAT | 86 |
| | CCCCGATTAcaAGAA | |
| 8 | ACCCcgCCGACATCCCCGATTACAAGAAactgactgCCCGCCGACA | 87 |
| | TCCCCGATTAcaAGAA | |
| 10 | ACCCcgCCGACATCCCCGATTACAAGAAactgactgacCCCGCCGAC | 88 |
| | ATCCCCGATTAcaAGAA | |
| 11 | ACCCcgCCGACATCCCCGATTACAAGAAactgactgacgCCCGCCGA | 89 |
| | CATCCCCGATTAcaAGAA | |
| 16 | ACCCcgCCGACATCCCCGATTACAAGAAactgactgactgactgCCCGC | 90 |
| | CGACATCCCCGATTAcaAGAA | |
| Gap | Sa-ST1 “PAM in-out” target sequence | 91 |
| 0 | TTCTTGTAATCGGGGATGTCGGcgGGGTCCCGCCGACATCCCC | 92 |
| | GATTAcaAGAA | |
| 2 | TTCTTGTAATCGGGGATGTCGGcgGGGTacCCCGCCGACATCCC | 93 |
| | CGATTAcaAGAA | |
| 3 | TTCTTGTAATCGGGGATGTCGGcgGGGTacgCCCGCCGACATCC | 94 |
| | CCGATTAcaAGAA | |
| 4 | TTCTTGTAATCGGGGATGTCGGcgGGGTactgCCCGCCGACATC | 95 |
| | CCCGATTAcaAGAA | |
| 5 | TTCTTGTAATCGGGGATGTCGGcgGGGTactgaCCCGCCGACATC | 96 |
| | CCCGATTAcaAGAA | |
| 6 | TTCTTGTAATCGGGGATGTCGGcgGGGTactgacCCCGCCGACAT | 97 |
| | CCCCGATTAcaAGAA | |
| 8 | TTCTTGTAATCGGGGATGTCGGcgGGGTactgactgCCCGCCGACA | 98 |
| | TCCCCGATTAcaAGAA | |
| 10 | TTCTTGTAATCGGGGATGTCGGcgGGGTactgactgacCCCGCCGAC | 99 |
| | ATCCCCGATTAcaAGAA | |
| 11 | TTCTTGTAATCGGGGATGTCGGcgGGGTactgactgacgCCCGCCGA | 100 |
| | CATCCCCGATTAcaAGAA | |
| 16 | TTCTTGTAATCGGGGATGTCGGcgGGGTactgactgactgactgCCCGCC | 101 |
| | GACATCCCCGATTAcaAGAA | |

TABLE 4
List of protein sequence

Name: TALE 1L-SceVmaCt-VP64
Keys: HA-tag, TALE 1L, SceVmaCt, VP64
SEQ ID NO: 102
MYPYDVPDYAGPKKKRKVDLRTLGYSQQQQEKIKPKVRSTVAQHHEALVGHGFT
HAHIVALSQHPAALGTVAVTYQHIITALPEATHEDIVGVGKQWSGARALEALLTD
AGELRGPPLQLDTGQLVKIAKRGGVTAMEAVHASRNALTGAPLNLTPDQVVAIA
SNNGGKQALETVQRLLPVLCQDHGLTPDQVVAIASNIGGKQALETVQRLLPVLC
QDHGLTPDQVVAIASNIGGKQALETVQRLLPVLCQDHGLTPDQVVAIASNNGGK
QALETVQRLLPVLCQDHGLTPDQVVAIASNIGGKQALETVQRLLPVLCQDHGLT
PDQVVAIASNIGGKQALETVQRLLPVLCQDHGLTPDQVVAIASNNGGKQALETV
QRLLPVLCQDHGLTPDQVVAIASHDGGKQALETVQRLLPVLCQDHGLTPDQVV
AIASNGGGKQALETVQRLLPVLCQDHGLTPDQVVAIASNNGGKQALETVQRLLP
VLCQDHGLTPDQVVAIASHDGGKQALETVQRLLPVLCQDHGLTPDQVVAIASNI
GGKQALETVQRLLPVLCQDHGLTPDQVVAIASNNGGKQALETVQRLLPVLCQD
HGLTPDQVVAIASNNGGKQALETVQRLLPVLCQDHGLTPDQVVAIASNIGGKQA
LETVQRLLPVLCQDHGLTPDQVVAIASNNGGKQALETVQRLLPVLCQDHGLTPD
QVVAIASNNGGKQALETVQRLLPVLCQDHGLTPDQVVAIASNGGGKQALESIVA
QLSRPDPALAALTNDHLVALACLGGRPAMDAVKKGLPHAPELIRRVNRRIGERT
SHRVALRGSGGGSGGGSGGGSGGGSGGGSGGGSVLLNVLSKCAGSKKFRPAPAAAF
ARECRGFYFELQELKEDDYYGITLSDDSDHQFLLANQVVVHNCTMTEKGSGGRADA
LDDFDLDMLGSDALDDFDLDMLGSDALDDFDLDMLGSDALDDFDLDMLINC
Name: ZF9-SceVmaNt-TALE 1R
Keys: Flag tag, ZF9, SceVmaNt, TALE 1R
SEQ ID NO: 103
MDYKDDDDKPKKKRKVSRPGERPFCRICMRNFSDKTKLRVHTRTHTGEKPFCRIC
MRNFSVRHNLTRHLRTHTGEKPFQCRICMRNFSQSTSLQRHLKTHLRGFGGVLEKGC
FAKGTNVLMADGSIECIENIEVGNKVMGKDGRPREVIKLPRGRETMYSVVQKSQHRA
HKSDSSREVPELLKFTCNATHELVVRTPRSVRRLSRTIKGVEYFEVITFEMGQKKAPD
GRIVELVKEVSKSYPISEGPERANELVESYRKASNKAYFEWTIEARDLSLLGCHVRKA
TYQTYAPIGGGSGGGSGGGSGGGSGGGSGGGSTQVDLRTLGYSQQQQEKIKPKVR
STVAQHHEALVGHGFTHAHIVALSQHPAALGTVAVTYQHIITALPEATHEDIVGV
GKQWSGARALEALLTDAGELRGPPLQLDTGQLVKIAKRGGVTAMEAVHASRNA
LTGAPLNLTPDQVVAIASNNGGKQALETVQRLLPVLCQDHGLTPDQVVAIASNN
GGKQALETVQRLLPVLCQDHGLTPDQVVAIASNIGGKQALETVQRLLPVLCQDH
GLTPDQVVAIASNNGGKQALETVQRLLPVLCQDHGLTPDQVVAIASNNGGKQAL
ETVQRLLPVLCQDHGLTPDQVVAIASNNGGKQALETVQRLLPVLCQDHGLTPDQ
VVAIASNNGGKQALETVQRLLPVLCQDHGLTPDQVVAIASNIGGKQALETVQRL
LPVLCQDHGLTPDQVVAIASNIGGKQALETVQRLLPVLCQDHGLTPDQVVAIASN
NGGKQALETVQRLLPVLCQDHGLTPDQVVAIASNGGGKQALETVQRLLPVLCQ
DHGLTPDQVVAIASNNGGKQALETVQRLLPVLCQDHGLTPDQVVAIASNNGGKQ
ALETVQRLLPVLCQDHGLTPDQVVAIASNGGGKQALETVQRLLPVLCQDHGLTP
DQVVAIASHDGGKQALESIVAQLSRPDPALAALTNDHLVALACLGGRPAMDAVK
KGLPHAPELIRRVNRRIGERTSHRVA
Name: TALE 1R-SceVmaCt, VP64
Keys: HA tag, TALE 1R, SceVmaCt, VP64
SEQ ID NO: 104
MYPYDVPDYAGPKKKRKVDLRTLGYSQQQQEKIKPKVRSTVAQHHEALVGHGFT
HAHIVALSQHPAALGTVAVTYQHIITALPEATHEDIVGVGKQWSGARALEALLTD
AGELRGPPLQLDTGQLVKIAKRGGVTAMEAVHASRNALTGAPLNLTPDQVVAIA
SNNGGKQALETVQRLLPVLCQDHGLTPDQVVAIASNNGGKQALETVQRLLPVLC
QDHGLTPDQVVAIASNIGGKQALETVQRLLPVLCQDHGLTPDQVVAIASNNGGK
QALETVQRLLPVLCQDHGLTPDQVVAIASNNGGKQALETVQRLLPVLCQDHGLT
PDQVVAIASNNGGKQALETVQRLLPVLCQDHGLTPDQVVAIASNNGGKQALETV
QRLLPVLCQDHGLTPDQVVAIASNIGGKQALETVQRLLPVLCQDHGLTPDQVVA
IASNIGGKQALETVQRLLPVLCQDHGLTPDQVVAIASNNGGKQALETVQRLLPVL
CQDHGLTPDQVVAIASNGGGKQALETVQRLLPVLCQDHGLTPDQVVAIASNNGG
KQALETVQRLLPVLCQDHGLTPDQVVAIASNNGGKQALETVQRLLPVLCQDHG
LTPDQVVAIASNGGGKQALETVQRLLPVLCQDHGLTPDQVVAIASHDGGKQALE
SIVAQLSRPDPALAALTNDHLVALACLGGRPAMDAVKKGLPHAPELIRRVNRRIG
ERTSHRVALRGSGGGSGGGSGGGSGGGSGGGSGGGSVLLNVLSKCAGSKKFRPAPA
AAFARECRGFYFELQELKEDDYYGITLSDDSDHQFLLANQVVVHNCTMTEKGSGGRA
DALDDFDLDMLGSDALDDFDLDMLGSDALDDFDLDMLGSDALDDFDLDMLINC
Name: Flag-ZF9-SceVmaNt-TALE 1L
Keys: Flag-ZF9, SceVmaNt, TALE 1L
SEQ ID NO: 105
MDYKDDDDKPKKKRKVSRPGERPFCRICMRNFSDKTKLRVHTRTHTGEKPFCRIC
MRNFSVRHNLTRHLRTHTGEKPFQCRICMRNFSQSTSLQRHLKTHLRGFGGVLEKGC
FAKGTNVLMADGSIECIENIEVGNKVMGKDGRPREVIKLPRGRETMYSVVQKSQHRA
HKSDSSREVPELLKFTCNATHELVVRTPRSVRRLSRTIKGVEYFEVITFEMGQKKAPD
GRIVELVKEVSKS YPISEGPERANELVES YRKASNKAYFEWTIEARDLSLLGCHVRKA
TYQTYAPIGGGSGGGSGGGSGGGSGGGSGGGSTQVDLRTLGYSQQQQEKIKPKVR
STVAQHHEALVGHGFTHAHIVALSQHPAALGTVAVTYQHIITALPEATHEDIVGV
GKQWSGARALEALLTDAGELRGPPLQLDTGQLVKIAKRGGVTAMEAVHASRNA
LTGAPLNLTPDQVVAIASNNGGKQALETVQRLLPVLCQDHGLTPDQVVAIASNIG
GKQALETVQRLLPVLCQDHGLTPDQVVAIASNIGGKQALETVQRLLPVLCQDHG
LTPDQVVAIASNNGGKQALETVQRLLPVLCQDHGLTPDQVVAIASNIGGKQALE
TVQRLLPVLCQDHGLTPDQVVAIASNIGGKQALETVQRLLPVLCQDHGLTPDQV
VAIASNNGGKQALETVQRLLPVLCQDHGLTPDQVVAIASHDGGKQALETVQRLL
PVLCQDHGLTPDQVVAIASNGGGKQALETVQRLLPVLCQDHGLTPDQVVAIASN
NGGKQALETVQRLLPVLCQDHGLTPDQVVAIASHDGGKQALETVQRLLPVLCQ
DHGLTPDQVVAIASNIGGKQALETVQRLLPVLCQDHGLTPDQVVAIASNNGGKQ
ALETVQRLLPVLCQDHGLTPDQVVAIASNNGGKQALETVQRLLPVLCQDHGLTP
DQVVAIASNIGGKQALETVQRLLPVLCQDHGLTPDQVVAIASNNGGKQALETVQ
RLLPVLCQDHGLTPDQVVAIASNNGGKQALETVQRLLPVLCQDHGLTPDQVVAI
ASNGGGKQALESIVAQLSRPDPALAALTNDHLVALACLGGRPAMDAVKKGLPH
APELIRRVNRRIGERTSHRVA
Name: Sa dCas9-SceVmaCt-VP64
Keys: HA tag, Sa dCas9, SceVmaCt, YP64
SEQ ID NO: 106
MYPYDVPDYAGSLAPKKKRKVGIHGVPAAKRNYILGLAIGITSVGYGIIDYETRDVI
DAGVRLFKEANVENNEGRRSKRGARRLKRRRRHRIQRVKKLLFDYNLLTDHSEL
SGINPYEARVKGLSQKLSEEEFSAALLHLAKRRGVHNVNEVEEDTGNELSTKEQI
SRNSKALEEKYVAELQLERLKKDGEVRGSINRFKTSDYVKEAKQLLKVQKAYHQ
LDQSFIDTYIDLLETRRTYYEGPGEGSPFGWKDIKEWYEMLMGHCTYFPEELRS
VKYAYNADLYNALNDLNNLVITRDENEKLEYYEKFQIIENVFKQKKKPTLKQIAK
EILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQI
AIFNRLKLVPKKVDLSQQKEIPTTLVDDFILSPVVKRSFIQSIKVINAIIKKYGLPND
IIIELAREKNSKDAQKMINEMQKRNRQTNERIEEIIRTTGKENAKYLIEKIKLHDM
QEGKCLYSLEAIPLEDLLNNPFNYEVDHIIPRSVSFDNSFNNKVLVKQEEASKKGN
RTPFQYLSSSDSKISYETFKKHILNLAKGKGRISKTKKEYLLEERDINRFSVQKDFI
NRNLVDTRYATRGLMNLLRSYFRVNNLDVKVKSINGGFTSFLRRKWKFKKERN
KGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNN
LNGLYDKDNDKLKKLINKSPEKLLMYHHDPQTYQKLKLIMEQYGDEKNPLYKY
YEETGNYLTKYSKKDNGPVIKKIKYYGNKLNAHLDITDDYPNSRNKVVKLSLKP
YRFDVYLDNGVYKFVTVKNLDVIKKENYYEVNSKCYEEAKKLKKISNQAEFIASF
YNNDLIKINGELYRVIGVNNDLLNRIEVNMIDITYREYLENMNDKRPPRIIKTIASK
TQSIKKYSTDILGNLYEVKSKKHPQIIKKGKRPAATKKAGQAKKKKGSMRGSGGG
SGGGSGGGSGGGSGGGSGGGSVLLNVLSKCAGSKKFRPAPAAAFARECRGFYFELQE
LKEDDYYGITLSDDSDHQFLLANQVVVHNCTMTEKGS GGRADALDDFDLDMLGSDA
LDDFDLDMLGSDALDDFDLDMLGSDALDDFDLDMLINC
Name: ZF9-SceVmaNt-Nm dCas9
Keys: Flag tag, ZF9, SceVmaNt, Nm dCas9
SEQ ID NO: 107
MDYKDDDDKPKKKRKVSRPGERPFCRICMRNFSDKTKLRVHTRTHTGEKPFCRIC
MRNFSVRHNLTHRLRTHTGEKPFQCRICMRNFSQSTSLGRHLKTHLRGFGGVLEKGC
FAKGTNVLMADGSIECIENIEVGNKVMGKDGRPREVIKLPRGRETMYSVVQKSQHRA
HKSDSSREVPELLKFTCNATHELVVRTPRSVRRLSRTIKGVEYFEVITFEMGQKKAPD
GRIVELVKEVSKSYPISEGPERANELVESYRKASNKAYFEWTIEARDLSLLGCHVRKA
TYQTYAPIGGGSGGGSGGGSGGGSGGGSGGGSTLMAAFKPNPINYILGLAIGIASVG
WAMVEIDEDENPICLIDLGVRVFERAEVPKTGDSLAMARRLARSVRRLTRRRAH
RLLRARRLLKREGVLQAADFDENGLIKSLPNTPWQLRAAALDRKLTPLEWSAVL
LHLIKHRGYLSQRKNEGETADKELGALLKGVADNAHALQTGDFRTPAELALNK
FEKESGHIRNQRGDYSHTFSRKDLQAELILLFEKQKEFGNPHVSGGLKEGIETLL
MTQRPALSGDAVQKMLGHCTFEPAEPKAAKNTYTAERFIWLTKLNNLRILEQGS
ERPLTDTERATLMDEPYRKSKLTYAQARKLLGLEDTAFFKGLRYGKDNAEASTL
MEMKAYHAISRALEKEGLKDKKSPLNLSPELQDEIGTAFSLFKTDEDITGRLKDR
IQPEILEALLKHISFDKFVQISLKALRRIVPLMEQGKRYDEACAEIYGDHYGKKNT
EEKIYLPPIPADEIRNPVVLRALSQARKVINGVVRRYGSPARIHIETAREVGKSFKD
RKEIEKRQEENRKDREKAAAKFREYFPNFVGEPKSKDILKLRLYEQQHGKCLYS
GKEINLGRLNEKGYVEIAAALPFSRTWDDSFNNKVLVLGSEAQNKGNQTPYEYF
NGKDNSREWQEFKARVETSRFPRSKKQRILLQKFDEDGFKERNLNDTRYVNRFL
CQFVADRMRLTGKGKKRVFASNGQITNLLRGFWGLRKVRAENDRHHALDAVV
VACSTVAMQQKITRFVRYKEMNAFDGKTIDKETGEVLHQKTHFPQPWEFFAQE
VMIRVFGKPDGKPEFEEADTPEKLRTLLAEKLSSRPEAVHEYVTPLFVSRAPNRK
MSGQGHMETVKSAKRLDEGVSVLRVPLTQLKLKDLEKMVNREREPKLYEALKA
RLEAHKDDPAKAFAEPFYKYDKAGNRTQQVKAVRVEQVQKTGVWVRNHNGIA
DNATMVRVDVFEKGDKYYLVPIYSWQVAKGILPDRAVVQGKDEEDWQLIDDSF
NFKFSLHPNDLVEVITKKARMFGYFASCHRGTGNINIRIHDLDHKIGKNGILEGIG
VKTALSFQKYQIDELGKEIRPCRLKKRPPVRSRADPKKKRKV
NAME: ZF9-VmaNt-ST1 dCas9
Keys: Flag tag, ZF9, SceVmaNt, ST1 dCas9
SEQ ID NO: 108
MDYKDDDDKPKKKRKVSRPGERPFCRICMRNFSDKTKLRVHTRTHTGEKPFCRIC
MRNFSVRHNLTRHLRTHTGEKPFQCRICMRNFSQSTSLQRHLKTHLRGFGGVLEKGC
FAKGTNVLMADGSIECIENIEVGNKVMGKDGRPREVIKLPRGRETMYSVVQKSQHRA
HKSDSSREVPELLKFTCNATHELVVRTPRSVRRLSRTIKGVEYFEVITFEMGQKKAPD
GRIVELVKEVSKSYPISEGPERANELVESYRKASNKAYFEWTIEARDLSLLGCHVRKA
TYQTYAPIGGGSGGGSGGGSGGGSGGGSGGGSTLMSDLVLGLAIGIGSVGVGILNK
VTGEIIHKNSRIFPAAQAENNLVRRTNRQGRRLARRKKHRRVRLNRLFEESGMT
DFTKISINLNPYQLRVKGLTDELSNEELFIALKNMVKHRGISYLDDASDDGNSSVG
DYAQIVKENSKQLETKTPGQIQLERYQTYGQLRGDFTVEKDGKKHRLINVFPTS
AYRSEALRILQTQQEFNPQITDEFINRYLEILTGKRKYYHGPGNEKSRTDYGRYR
TSGETLDNIFGILIGKCTFYPDEFRAAKASYTAQEFNLLNDLNNLTVPTETKKLSK
EQKNQIINYVKNEKAMGPAKLFKYIAKLLSCDVADIKGYRIDKSGKAEIHTFEAY
RKMKTLETLDIEQMDRETLDKLAYVLTLNTEREGIQEALEHEFADGSFSQKQVD
ELVQFRKANSSIFGKGWHNFSVKLMMELIPELYETSEEQMTILTRLGKQKTTSSS
NKTKYIDEKLLTEEIYNPVVAKSVRQAIKIVNAAIKEYGDFDNIVIEMARETNEDD
EKKAIQKIQKANKDEKDAAMLKAANQYNGKAELPHSVFHGHKQLATKIRLWH
QQGERCLYTGKTISIHDLINNSNQFEVAAILPLSITFDDSLANKVLVYATAAQEKG
QRTPYQALDSMDDAWSFRELKAFVRESKTLSNKKKEYLLTEEDISKFDVRKKFIE
RNLVDTRYASRVVLNALQEHFRAHKIDTKVSVVRGQFTSQLRRHWGIEKTRDTY
HHHAVDALIIAASSQLNLWKKQKNTLVSYSEDQLLDIETGELISDDEYKESVFKAP
YQHFVDTLKSKEFEDSILFSYQVDSKFNRKISDATIYATRQAKVGKDKADETYVL
GKIKDIYTQDGYDAFMKIYKKDKSKFLMYRHDPQTFEKVIEPILENYPNKQINDK
GKEVPCNPFLKYKEEHGYIRKYSKKGNGPEIKSLKYYDSKLGNHIDITPKDSNNK
VVLQSVSPWRADVYFNKTTGKYEILGLKYADLQFDKGTGTYKISQEKYNDIKKK
EGVDSDSEFKFTLYKNDLLLVKDTETKEQQLFRFLSRTMPKQKHYVELKPYDKQ
KFEGGEALIKVLGNVANSGQCKKGLGKSNISIYKVRTDVLGNQHIIKNEGDKPKL
DFSRADPKKKRKV
Name: Nm dCas9-VmaCt-VP64
Keys: HA tag, Nm dCas9, SceVmaCt, VP64
SEQ ID NO: 109
MYPYDVPDYAGSLAAFKPNPINYILGLAIGIASVGWAMVEIDEDENPICLIDLGVR
VFERAEVPKTGDSLAMARRLARSVRRLTRRRAHRLLRARRLLKREGVLQAADF
DENGLIKSLPNTPWQLRAAALDRKLTPLEWSAVLLHLIKHRGYLSQRKNEGETA
DKELGALLKGVADNAHALQTGDFRTPAELALNKFEKESGHIRNQRGDYSHTFSR
KDLQAELILLFEKQKEFGNPHVSGGLKEGIETLLMTQRPALSGDAVQKMLGHCT
FEPAEPKAAKNTYTAERFIWLTKLNNLRILEQGSERPLTDTERATLMDEPYRKSK
LTYAQARKLLGLEDTAFFKGLRYGKDNAEASTLMEMKAYHAISRALEKEGLKD
KKSPLNLSPELQDEIGTAFSLFKTDEDITGRLKDRIQPEILEALLKHISFDKFVQISL
KALRRIVPLMEQGKRYDEACAEIYGDHYGKKNTEEKIYLPPIPADEIRNPVVLRA
LSQARKVINGVVRRYGSPARIHIETAREVGKSFKDRKEIEKRQEENRKDREKAAA
KFREYFPNFVGEPKSKDILKLRLYEQQHGKCLYSGKEINLGRLNEKGYVEIAAAL
PFSRTWDDSFNNKVLVLGSEAQNKGNQTPYEYFNGKDNSREWQEFKARVETSRF
PRSKKQRILLQKFDEDGFKERNLNDTRYVNRFLCQFVADRMRLTGKGKKRVFA
SNGQITNLLRGFWGLRKVRAENDRHHALDAVVVACSTVAMQQKITRFVRYKEM
NAFDGKTIDKETGEVLHQKTHFPQPWEFFAQEVMIRVFGKPDGKPEFEEADTPE
KLRTLLAEKLSSRPEAVHEYVTPLFVSRAPNRKMSGQGHMETVKSAKRLDEGVS
VLRVPLTQLKLKDLEKMVNREREPKLYEALKARLEAHKDDPAKAFAEPFYKYD
KAGNRTQQVKAVRVEQVQKTGVWVRNHNGIADNATMVRVDVFEKGDKYYLVP
IYSWQVAKGILPDRAVVQGKDEEDWQLIDDSFNFKFSLHPNDLVEVITKKARMF
GYFASCHRGTGNINIRIHDLDHKIGKNGILEGIGVKTALSFQKYQIDELGKEIRPC
RLKKRPPVRSRADPKKKRKVMRGSGGGSGGGSGGGSGGGSGGGSGGGSVLLNVLS
KCAGSKKFRPAPAAAFARECRGFYFELQELKEDDYYGITLSDDSDHQFLLANQVVVH
NCTMTEKGSGGRADALDDFDLDMLGSDALDDFDLDMLGSDALDDFDLDMLGSDAL
DDFDLDMLINC
Name: ZF9-SceVmaNt-Sa dCas9
Keys: Flag tag, ZF9, SceVmaNt, Sa dCas9
SEQ ID NO: 110
MDYKDDDDKPKKKRKVSRPGERPFCRICMRNFSDKTKLRVHTRTHTGEKPFCRIC
MRNFSVRHNLTRHLRTHTGEKPFQCRICMRNFSQSTSLQRHLKTHLRGFGGVLEKGC
FAKGTNVLMADGSIECIENIEVGNKVMGKDGRPREVIKLPRGRETMYSVVQKSQHRA
HKSDSSREVPELLKFTCNATHELVVRTPRSVRRLSRTIKGVEYFEVITFEMGQKKAPD
GRIVELVKEVSKSYPISEGPERANELVESYRKASNKAYFEWTIEARDLSLLGCHVRKA
TYQTYAPIGGGSGGGSGGGSGGGSGGGSGGGSTLLAPKKKRKVGIHGVPAAKRNYIL
GLAIGITSVGYGIIDYETRDVIDAGVRLFKEANVENNEGRRSKRGARRLKRRRRH
RIQRVKKLLFDYNLLTDHSELSGINPYEARVKGLSQKLSEEEFSAALLHLAKRRG
VHNVNEVEEDTGNELSTKEQISRNSKALEEKYVAELQLERLKKDGEVRGSINRFK
TSDYVKEAKQLLKVQKAYHQLDQSFIDTYIDLLETRRTYYEGPGEGSPFGWKDI
KEWYEMLMGHCTYFPEELRSVKYAYNADLYNALNDLNNLVITRDENEKLEYYE
KFQIIENVFKQKKKPTLKQIAKEILVNEEDIKGYRVTS TGKPEFTNLKVYHDIKDI
TARKEIIENAELLDQIAKILTIYQSSEDIQEELTNLNSELTQEEIEQISNLKGYTGTH
NLSLKAINLILDELWHTNDNQIAIFNRLKLVPKKVDLS QQKEIPTTLVDDFILSPVV
KRSFIQSIKVINAIIKKYGLPNDIIIELAREKNSKDAQKMINEMQKRNRQTNERIEE
IIRTTGKENAKYLIEKIKLHDMQEGKCLYSLEAIPLEDLLNNPFNYEVDHIIPRSVS
FDNSFNNKVLVKQEEASKKGNRTPFQYLSSSDSKISYETFKKHILNLAKGKGRISK
TKKEYLLEERDINRFSVQKDFINRNLVDTRYATRGLMNLLRSYFRVNNLDVKVK
SINGGFTSFLRRKWKFKKERNKGYKHHAEDALIIANADFIFKEWKKLDKAKKV
MENQMFEEKQAESMPEIETEQEYKEIFITPHQIKHIKDFKDYKYSHRVDKKPNRE
LINDTLYSTRKDDKGNTLIVNNLNGLYDKDNDKLKKLINKSPEKLLMYHHDPQT
YQKLKLIMEQYGDEKNPLYKYYEETGNYLTKYSKKDNGPVIKKIKYYGNKLNA
HLDITDDYPNSRNKVVKLSLKPYRFDVYLDNGVYKFVTVKNLDVIKKENYYEVN
SKCYEEAKKLKKISNQAEFIASFYNNDLIKINGELYRVIGVNNDLLNRIEVNMIDIT
YREYLENMNDKRPPRIIKTIASKTQSIKKYS TDILGNLYEVKSKKHPQIIKKGKRP
AATKKAGQAKKKKGS

### REFERENCES

1. Slomovic, S. and J. J. Collins, DNA sense-and-respond protein modules for mammalian cells. Nat Methods, 2015. 12(11): p. 1085-90.
2. Hossain, M. A., et al., Artificial zinc finger DNA binding domains: versatile tools for genome engineering and modulation of gene expression. J Cell Biochem, 2015. 116(11): p. 2435-44.
3. Boch, J., et al., Breaking the code of DNA binding specificity of TAL-type III effectors. Science, 2009. 326(5959): p. 1509-12.
4. Moscou, M. J. and A. J. Bogdanove, A simple cipher governs DNA recognition by TAL effectors. Science, 2009. 326(5959): p. 1501.
5. Cermak, T., et al., Efficient design and assembly of custom TALEN and other TAL effector-based constructs for DNA targeting. Nucleic Acids Res, 2011. 39(12): p. e82.
6. Ran, F. A., et al., In vivo genome editing using Staphylococcus aureus Cas9. Nature, 2015. 520(7546): p. 186-91.
7. Esvelt, K. M., et al., Orthogonal Cas9 proteins for RNA-guided gene regulation and editing. Nat Methods, 2013. 10(11): p. 1116-21.
8. Hou, Z., et al., Efficient genome engineering in human pluripotent stem cells using Cas9 from Neisseria meningitidis. Proc Natl Acad Sci USA, 2013. 110(39): p. 15644-9
9. Choi, P. S. and Meyerson, M. (2014) Targeted genomic rearrangements using CRISPR/Cas technology. Nature communications 5.
10. Torres, R., Martin, M., Garcia, A., Cigudosa, J. C., Ramirez, J., and Rodriguez-Perales, S. (2014) Engineering human tumour-associated chromosomal translocations with the RNA-guided CRISPR-Cas9 system. Nature communications 5: 3964.
11. Cheng, A. W., Jillette, N., Lee, P., Plaskon, D., Fujiwara, Y., Wang, W., Taghbalout, A., and Wang, H. (2016) Casilio: a versatile CRISPR-Cas9-Pumilio hybrid for gene regulation and genomic labeling. Cell research.
12. Topilina, N. I. and Mills, K. V. (2014) Recent advances in in vivo applications of intein-mediated protein splicing. Mobile Dna 5(1): 5.
13. Gregoire, D. and Kmita, M. (2014) Genetic cell ablation. Mouse Molecular Embryology: Methods and Protocols: 421-436.
14. Chelur, D. S. and Chalfie, M. (2007) Targeted cell killing by reconstituted caspases. Proceedings of the National Academy of Sciences 104(7): 2283-2288.
15. Grohmann, M., Paulmann, N., Fleischhauer, S., Vowinckel, J., Priller, J., and Walther, D. J. (2009) A mammalianized synthetic nitroreductase gene for high-level expression. BMC cancer 9(1): 301.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

What is claimed is:

1. A sequence detector system comprising:
a first guide RNA (gRNA) and a first catalytically-inactive RNA-guided nuclease linked to an N-terminal fragment of an intein, wherein the N-terminal fragment is linked to a first polypeptide, and the first gRNA is engineered to bind to a first target sequence; and
a second gRNA and a second catalytically-inactive RNA-guided nuclease linked to an C-terminal fragment of an intein, wherein the C-terminal fragment is linked to a second polypeptide, and the second gRNA is engineered to bind to a second target sequence adjacent to the first target sequence,
wherein the first and second catalytically-inactive RNA-guided nucleases are orthogonal to each other.

2. The sequence detector system of claim 1, wherein the N-terminal fragment and the C-terminal fragment of the intein catalyze joining of the first polypeptide to the second polypeptide.

3. The sequence detector system of claim 1, wherein the first and second catalytically-inactive RNA-guided nucleases are selected from catalytically-inactive Cas nucleases and catalytically-inactive Cpf1 nucleases.

4. The sequence detector system of claim 3, wherein the first and second catalytically-inactive RNA-guided nucleases are selected from catalytically-inactive Streptococcus thermophiles Cas9 nuclease, Staphylococcus aureus Cas9 nucleases and Neisseria meningitidis Cas9 nucleases.

5. The sequence detector system of claim 4, wherein the first catalytically-inactive RNA-guided nuclease is a catalytically-inactive Streptococcus thermophiles Cas9 nuclease and the second catalytically-inactive RNA-guided nuclease is a catalytically-inactive Neisseria meningitidis Cas9 nuclease.

6. The sequence detector system of claim 1, wherein the intein is an engineered split intein or a naturally-occurring split intein.

7. The sequence detector system of claim 6, wherein the intein is selected from Saccharomyces cerevisiae VMA (Sce VMA) split inteins, Synechocystis sp. DnaB (Ssp DnaB) split inteins, Synechocystis sp. GyrB (Ssp GyrB) split inteins, Synechocystis sp. DnaE (Ssp DnaE) split inteins, and Nostoc punctiforme DnaE (Npu DnaE) split inteins.

8. The sequence detector system of claim 1, wherein
(a) the first polypeptide is a first reporter molecule and the second polypeptide is a second reporter molecule; or
(b) the first polypeptide is an N-terminal fragment of a reporter molecule and the second polypeptide is a C-terminal fragment of the reporter molecule.

9. The sequence detector of claim 8, wherein the first and/or second reporter molecule of (a) and/or the reporter molecule of (b) is selected from TagCFP, mTagCFP2, Azurite, ECFP2, mKalama1, Sirius, Sapphire, T-Sapphire, ECFP, Cerulean, SCFP3C, mTurquoise, mTurquoise2, monomeric Midoriishi-Cyan, TagCFP, mTFP1, EGFP, Emerald, Superfolder GFP, Monomeric Czami Green, TagGFP2, mUKG, mWasabi, Clover, mNeonGreen, EYFP, Citrine, Venus, SYFP2, TagYFP, Monomeric Kusabira-Orange, mKOκ, mKO2, mOrange, mOrange2, mRaspberry, mCherry, mStrawberry, mScarlet, mTangerine, tdTomato, TagRFP, TagRFP-T, mApple, mRuby, mRuby2, mPlum, HcRed-Tandem, mKate2, mNeptune, NirFP, TagRFP657, IFP1.4 and iRFP.

10. The sequence detector of claim 8, wherein the first and second reporter molecules of (a) are different from each other.

11. The sequence detector system of claim 1, wherein the first polypeptide is an N-terminal fragment of a toxic molecule and the second polypeptide is a C-terminal fragment of the toxic molecule.

12. The sequence detector of claim 11, wherein the toxic molecule is selected from toxins, pro-apoptotic proteins, and prodrug metabolic enzymes

13. The sequence detector system of claim 1, wherein
the first polypeptide is a first molecule of a synthetic transcription factor and the second polypeptide is a second molecule of the synthetic transcription factor; or
the first polypeptide is an N-terminal fragment of a synthetic transcription factor and the second polypeptide is a C-terminal fragment of the synthetic transcription factor.

14. The sequence detector system of claim 13, wherein the synthetic transcription factor binds to and activates transcription of a nucleic acid encoding a reporter molecule or a toxic molecule.

15. The sequence detector system of claim 14, wherein the nucleic acid encoding a reporter molecule or a toxic molecule comprises a minimal promoter and a binding site to which the synthetic transcription factor binds.

16. The sequence detector system of claim 1,
wherein the N terminus of the first catalytically-inactive RNA-guided nuclease is linked to the C terminus of the N-terminal fragment of the intein, the N terminus of the N-terminal fragment of the intein is linked to the C terminus of the first polypeptide, the C terminus of the second catalytically-inactive RNA-guided nuclease is linked to the N terminus of the C-terminal fragment of the intein, and the C terminus of the C-terminal fragment of the intein is linked to the N terminus of the second polypeptide.

17. A pair of engineered polynucleotides, wherein
the first polynucleotide of the pair encodes in the 5′ to 3′ direction a first polypeptide, an N-terminal fragment of an intein, a first catalytically-inactive RNA-guided nuclease, and
the second polynucleotide of the pair encodes in the 5′ to 3′ direction a second catalytically-inactive RNA-guided nuclease, a C-terminal fragment of the intein, and a second polypeptide,
wherein the first and second catalytically-inactive RNA-guided nucleases are orthogonal to each other.

18. A sequence detector system comprising:
a first TAL effector DNA-binding domain (TALE) linked to an N-terminal fragment of an intein, wherein the N-terminal fragment is linked to a first polypeptide, and the first TALE is engineered to bind to a first target sequence; and
a second TALE linked to an C-terminal fragment of an intein, wherein the C-terminal fragment is linked to a second polypeptide, and the second TALE is engineered to bind to a second target sequence adjacent to the first target sequence.

19. A pair of engineered polynucleotides, wherein
the first polynucleotide of the pair encodes in the 5′ to 3′ direction a first polypeptide, an N-terminal fragment of an intein, and a first TAL effector DNA-binding domain (TALE) engineered to bind to a first target sequence, and
the second polynucleotide of the pair encodes in the 5′ to 3′ direction a second TALE engineered to bind to a second target sequence adjacent to the first target sequence, a C-terminal fragment of the intein, and a second polypeptide.

20. A cell comprising: (a) the sequence detector system of claim 1 and (b) a genome comprising the first and second target sequences.

21. A cell comprising: (a) the sequence detector system of claim 18 and (b) a genome comprising the first and second target sequences.

22. A cell comprising: (a) the pair of engineered polynucleotides of claim 17 and (b) a genome comprising the first and second target sequences.

23. A cell comprising: (a) the pair of engineered polynucleotides of claim 19 and (b) a genome comprising the first and second target sequences.

24. A selective detection method comprising delivering to a population of cells the pair of engineered polynucleotides of claim 17, wherein the first and/or second polypeptide encodes a reporter molecule or a synthetic transcription factor that activates transcription of a nucleic acid encoding a reporter molecule, and assaying for expression or activity of the reporter molecule.

25. A selective detection method comprising delivering to a population of cells the pair of engineered polynucleotides of claim 19, wherein the first and/or second polypeptide encodes a reporter molecule or a synthetic transcription factor that activates transcription of a nucleic acid encoding a reporter molecule, and assaying for expression or activity of the reporter molecule.

26. A selective cell ablation method comprising delivering to a population of cells the pair of engineered polynucleotides of claim 17, wherein the first and/or second polypeptide encodes a toxic molecule or a synthetic transcription factor that activates transcription of a nucleic acid encoding a toxic molecule, and assaying for cell death.

27. A selective cell ablation method comprising delivering to a population of cells the pair of engineered polynucleotides of claim 19, wherein the first and/or second polypeptide encodes a toxic molecule or a synthetic transcription factor that activates transcription of a nucleic acid encoding a toxic molecule, and assaying for cell death.
